(19) United States
(12) Patent Application Publication
(10) Pub. No.: US 2025/0221142 A1
(43) Pub. Date: Jul. 3, 2025

(54) IMAGING ELEMENT AND METHOD OF MANUFACTURING IMAGING ELEMENT, AND LIGHT DETECTION DEVICE

(71) Applicant: SONY SEMICONDUCTOR SOLUTIONS CORPORATION, KANAGAWA (JP)

(72) Inventors: SHINTAROU HIRATA, KANAGAWA (JP); MASAHIRO JOEI, KANAGAWA (JP); IWAO YAGI, KANAGAWA (JP); KENICHI MURATA, KANAGAWA (JP)

(21) Appl. No.: 18/848,103

(22) PCT Filed: Mar. 7, 2023
(86) PCT No.: PCT/JP2023/008621

§ 371 Date: Sep. 17, 2024

(30) Foreign Application Priority Data
Mar. 25, 2022 (JP) 2022-050525

(51) Int. Cl. H10K 39/32; H10K 30/30; H10K 39/38

(52) U.S. Cl. CPC H10K 39/32; H10K 30/353; H10K 39/38

(57) ABSTRACT

An imaging element in one embodiment of the present disclosure includes a first electrode and a second electrode, a third electrode disposed opposed to the first electrode and the second electrode, a photoelectric conversion layer provided between the first electrode and the third electrode and between the second electrode and the third electrode, an insulating layer provided between the first electrode and the photoelectric conversion layer and between the second electrode and the photoelectric conversion layer and having an opening above the second electrode, a first layer provided between the photoelectric conversion layer and the insulating layer and formed at least above the first electrode, and a second layer formed at least above the second electrode while being electrically coupled to the second electrode via the opening and having a difference from the first layer in at least one of material composition, crystallinity, impurity concentration contained, or constituent element.

### TECHNICAL FIELD

The present disclosure relates, for example, to an imaging element using an organic material and a method of manufacturing the same, and to a light detection device.

### BACKGROUND ART

For example, PTL 1 discloses a solid-state imaging device in which a photoelectric conversion film and a semiconductor layer are provided between a first electrode and a second electrode disposed opposed to each other from the first electrode side, and in which a third electrode covered by an insulating film is further provided in the semiconductor layer.

### CITATION LIST

### Patent Literature

PTL1: WO2020/203249

### SUMMARY OF THE INVENTION

Meanwhile, for a light detection device that is used in an imaging device, etc., a captured image is expected to have an improved image quality.

It is desirable to provide an imaging element and a method of manufacturing an imaging element, and a light detection device that enable improvement of a captured image quality.

An imaging element according to one embodiment of the present disclosure includes: a first electrode and a second electrode; a third electrode disposed opposed to the first electrode and the second electrode; a photoelectric conversion layer provided between the first electrode and the third electrode and between the second electrode and the third electrode; an insulating layer provided between the first electrode and the photoelectric conversion layer and between the second electrode and the photoelectric conversion layer, the insulating layer having an opening above the second electrode; and a semiconductor layer including a first layer and a second layer, the first layer being provided between the photoelectric conversion layer and the insulating layer and formed at least above the first electrode, and the second layer being formed at least above the second electrode while being electrically coupled to the second electrode via the opening and having a difference from the first layer in at least one of material composition, crystallinity, impurity concentration contained, or constituent element.

One embodiment of the present disclosure provides a method of manufacturing an imaging element. The imaging element includes a first electrode and a second electrode, a third electrode disposed opposed to the first electrode and the second electrode, a photoelectric conversion layer provided between the first electrode and the third electrode and between the second electrode and the third electrode, an insulating layer provided between the first electrode and the photoelectric conversion layer and between the second electrode and the photoelectric conversion layer, the insulating layer having an opening above the second electrode, and a semiconductor layer including a first layer and a second layer, the first layer being provided between the photoelectric conversion layer and the insulating layer and formed at least above the first electrode, and the second layer being formed at least above the second electrode while being electrically coupled to the second electrode via the opening and having a difference from the first layer in at least one of material composition, crystallinity, impurity concentration contained, or constituent element. The method includes: forming the first layer using a physical vapor deposition method; and forming the second layer using an atomic layer deposition method.

A light detection device according to one embodiment of the present disclosure includes one or a plurality of imaging elements according to one embodiment of the present disclosure for each plurality of pixels.

In an imaging element according to one embodiment of the present disclosure, a method of manufacturing an imaging element according to one embodiment of the present disclosure, and a light detection device according to one embodiment of the present disclosure, a semiconductor layer including a first layer and a second layer is provided in which between an insulating layer covering, of a first electrode and a second electrode, the first electrode and having an opening above the second electrode and a photoelectric conversion layer, the first layer is formed at least above the first electrode, and the second layer is formed at least above the second electrode while being electrically coupled to the second electrode via the opening and having a difference from the first layer in at least one of material composition, crystallinity, impurity concentration contained, or constituent element. This improves a carrier transport characteristic to the second electrode.

### BRIEF DESCRIPTION OF DRAWINGS

FIG. 1 is a schematic cross-sectional view illustrating an example of a configuration of an imaging element according to one embodiment of the present disclosure.

FIG. 2 is a schematic plan view illustrating an example of a pixel configuration of an imaging device including the imaging element illustrated in FIG. 1.

FIG. 3A is a schematic cross-sectional view illustrating an example of a configuration of a photoelectric conversion section illustrated in FIG. 1.

FIG. 3B is a schematic cross-sectional view illustrating another example of the configuration of the photoelectric conversion section illustrated in FIG. 1.

FIG. 4 illustrates an energy level corresponding to a line A-A illustrated in FIG. 3A.

FIG. 5 illustrates an energy level corresponding to a line B-B illustrated in FIG. 3A.

FIG. 6 illustrates an energy level corresponding to a line C-C illustrated in FIG. 3A.

FIG. 7 is an equivalent circuit diagram of the imaging element illustrated in FIG. 1.

FIG. 8 is a schematic view illustrating an arrangement of a transistor included in a lower electrode and a control section in the imaging element illustrated in FIG. 1.

FIG. 9A is a cross-sectional view that describes a method of manufacturing the imaging element illustrated in FIG. 1.

FIG. 9B is a cross-sectional view illustrating a process following FIG. 9A.

FIG. 9C is a cross-sectional view illustrating a process following FIG. 9B.

FIG. 9D is a cross-sectional view illustrating a process following FIG. 9C.

FIG. 9E is a cross-sectional view illustrating a process following FIG. 9D.

FIG. 9F is a cross-sectional view illustrating a process following FIG. 9E.

FIG. 9G is a cross-sectional view illustrating a process following FIG. 9F.

FIG. 9H is a cross-sectional view illustrating a process following FIG. 9G.

FIG. 9I is a cross-sectional view illustrating a process following FIG. 9H.

FIG. 9J is a cross-sectional view illustrating a process following FIG. 9I.

FIG. 9K is a cross-sectional view illustrating a process following FIG. 9J.

FIG. 10 is a timing diagram illustrating an example of an operation of the imaging element illustrated in FIG. 1.

FIG. 11 is a schematic cross-sectional view illustrating a configuration of a photoelectric conversion section according to Modification example 1 of the present disclosure.

FIG. 12A is a cross-sectional view illustrating a method of manufacturing an imaging element illustrated in FIG. 11.

FIG. 12B is a cross-sectional view illustrating a process following FIG. 12A.

FIG. 12C is a cross-sectional view illustrating a process following FIG. 12B.

FIG. 12D is a cross-sectional view illustrating a process following FIG. 12C.

FIG. 13 is a schematic cross-sectional view illustrating a configuration of a photoelectric conversion section according to Modification example 2 of the present disclosure.

FIG. 14A is a cross-sectional view illustrating a method of manufacturing an imaging element illustrated in FIG. 13.

FIG. 14B is a cross-sectional view illustrating a process following FIG. 14A.

FIG. 14C is a cross-sectional view illustrating a process following FIG. 14B.

FIG. 14D is a cross-sectional view illustrating a process following FIG. 14C.

FIG. 14E is a cross-sectional view illustrating a process following FIG. 14D.

FIG. 14F is a cross-sectional view illustrating a process following FIG. 14E.

FIG. 14G is a cross-sectional view illustrating a process following FIG. 14F.

FIG. 14H is a cross-sectional view illustrating a process following FIG. 14G.

FIG. 14I is a cross-sectional view illustrating a process following FIG. 14H.

FIG. 15 is a schematic cross-sectional view illustrating a configuration of a photoelectric conversion section according to Modification example 3 of the present disclosure.

FIG. 16A is a cross-sectional view that describes a method of manufacturing an imaging element illustrated in FIG. 15.

FIG. 16B is a cross-sectional view illustrating a process following FIG. 16A.

FIG. 16C is a cross-sectional view illustrating a process following FIG. 16B.

FIG. 16D is a cross-sectional view illustrating a process following FIG. 16C.

FIG. 16E is a cross-sectional view illustrating a process following FIG. 16D.

FIG. 16F is a cross-sectional view illustrating a process following FIG. 16E.

FIG. 16G is a cross-sectional view illustrating a process following FIG. 16F.

FIG. 16H is a cross-sectional view illustrating a process following FIG. 16G.

FIG. 17A is a schematic cross-sectional view illustrating an example of a configuration of an imaging element according to Modification example 4 of the present disclosure.

FIG. 17B is a schematic plan view illustrating an example of a pixel configuration of an imaging device including the imaging element illustrated in FIG. 17A.

FIG. 18A is a schematic cross-sectional view illustrating an example of a configuration of an imaging element according to Modification example 5 of the present disclosure.

FIG. 18B is a schematic plan view illustrating an example of a pixel configuration of an imaging device including the imaging element illustrated in FIG. 18A.

FIG. 19 is a schematic cross-sectional view illustrating an example of a configuration of an imaging element according to Modification example 6 of the present disclosure.

FIG. 20 is a block diagram illustrating a configuration of an imaging device using, as a pixel, the imaging element illustrated in FIG. 1, etc.

FIG. 21 is a block diagram illustrating an example of a configuration of an electronic apparatus using the imaging device illustrated in FIG. 20.

FIG. 22A is a schematic view illustrating an example of an overall configuration of a light detection system using the imaging device illustrated in FIG. 20.

FIG. 22B illustrates an example of a circuit configuration of the light detection system illustrated in FIG. 22A.

FIG. 23 is a view depicting an example of a schematic configuration of an endoscopic surgery system.

FIG. 24 is a block diagram depicting an example of a functional configuration of a camera head and a camera control unit (CCU).

FIG. 25 is a block diagram depicting an example of schematic configuration of a vehicle control system.

FIG. 26 is a diagram of assistance in explaining an example of installation positions of an outside-vehicle information detecting section and an imaging section.

### MODES FOR CARRYING OUT THE INVENTION

In the following, some embodiments of the present disclosure will be described in detail with reference to the drawings. The embodiments described below are specific examples of the present disclosure, and the present disclosure is not limited to the following embodiments. In addition, the arrangement, dimensions, dimension ratios, and the like of components in the present disclosure are not limited to the embodiment illustrated in each drawing. It is to be noted that the description will be given in the following order.
1. Embodiment (Example of an imaging element including two types of semiconductor layers having at least one difference and provided above respective ones of a readout electrode and a storage electrode)
1-1. Configuration of Imaging Element
1-2. Method of Manufacturing Imaging Element
1-3. Signal Acquisition Operation by Imaging Element
1-4. Workings and Effects
2. Modification Examples
2-1. Modification Example 1 (Another example of layout of two types of semiconductor layers)
2-2. Modification Example 2 (Another example of layout of two types of semiconductor layers)
2-3. Modification Example 3 (Another example of layout of two types of semiconductor layers)
2-4. Modification Example 4 (Example of an imaging element that disperses light using a color filter)
2-5. Modification Example 5 (Another example of an imaging element that disperses light using a color filter)
2-6. Modification Example 6 (Example of an imaging element in which a plurality of photoelectric conversion sections are stacked)
3. Application Examples
4. Practical Applications

### 1. Embodiment

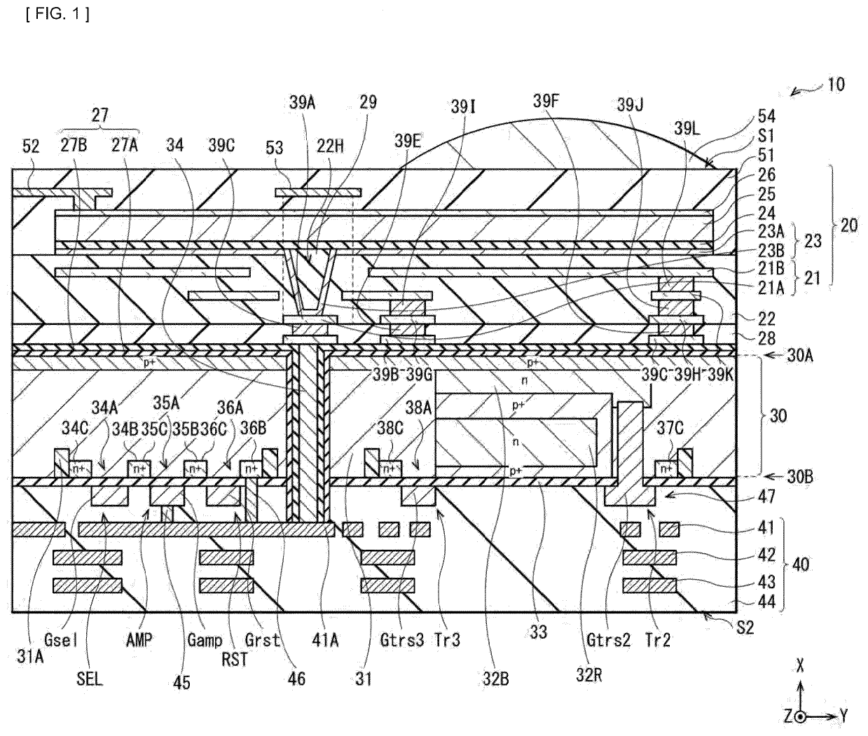

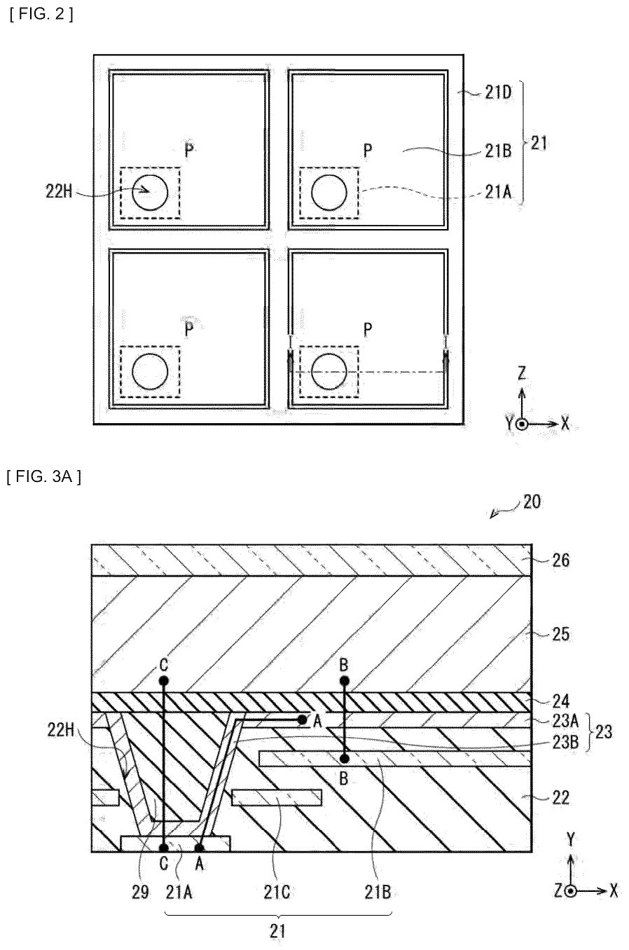

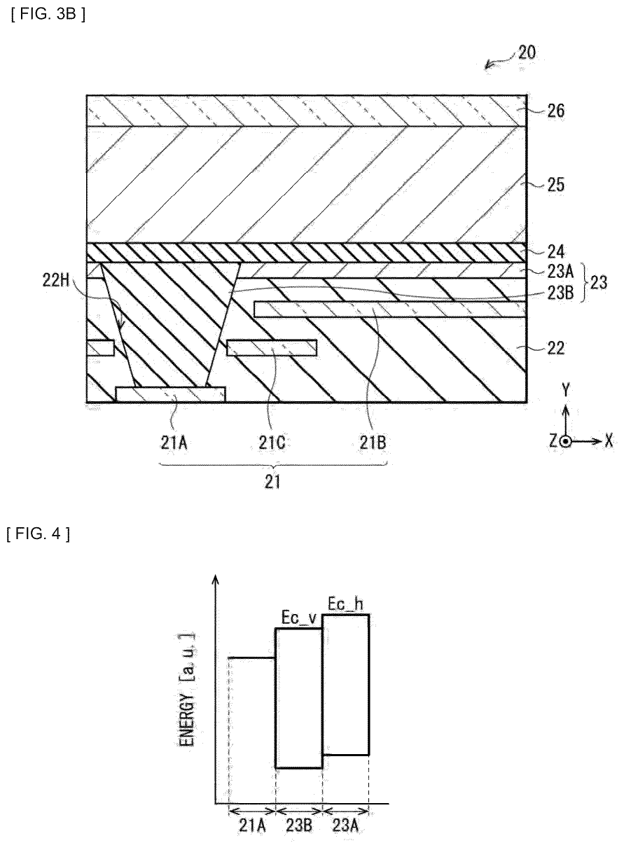

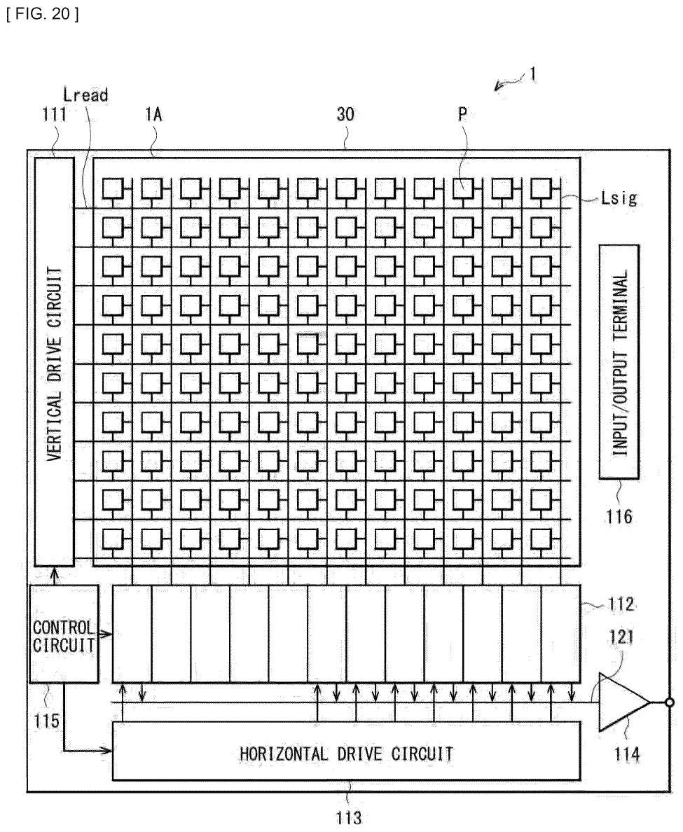

FIG. 1 illustrates a cross-sectional configuration of an imaging element (imaging element 10) according to one embodiment of the present disclosure. FIG. 2 schematically illustrates an example of a planar configuration of the imaging element 10 illustrated in FIG. 1, and FIG. 1 illustrates a cross section at a line I-I illustrated in FIG. 2. FIG. 3A is an enlarged view schematically illustrating an example of a cross-sectional configuration of a main section (photoelectric conversion section 20) of the imaging element 10 illustrated in FIG. 1. FIG. 3B is an enlarged view schematically illustrating another example of the cross-sectional configuration of the main section (photoelectric conversion section 20) of the imaging element 10 illustrated in FIG. 1. For example, the imaging element 10 is included in one pixel (unit pixel P) that is repeatedly arranged in an array in a pixel section 1A of an imaging device (for example, imaging device 1, see FIG. 20) such as a CMOS (Complementary Metal Oxide Semiconductor) image sensor, which is used in, for example, an electronic apparatus such as a digital still camera or video camera.

In the imaging element 10 of the present embodiment, the photoelectric conversion section 20 provided on a semiconductor substrate 30 includes, between a lower electrode 21 including a readout electrode 21A and a storage electrode 21B and a photoelectric conversion layer 25, a semiconductor layer 23 including a first layer 23A and a second layer 23B having a difference from each other above the readout electrode 21A and the storage electrode 21B, respectively. The difference between the first layer 23A and the second layer 23B is in at least one of material composition, crystallinity, impurity concentration contained, or constituent element. Between the lower electrode 21 and the semiconductor layer 23, an insulating layer 22 having an opening 22H on the readout electrode 21A is provided. The first layer 23A is provided above the storage electrode 21B via the insulating layer 22, and the second layer 23B is provided above the readout electrode 21A while being electrically coupled to the readout electrode 21A via the opening 22H. The readout electrode 21A corresponds to one specific example of a “second electrode” of the present disclosure, and the storage electrode 21B corresponds to one specific example of a “first electrode” of the present disclosure. In addition, the first layer 23A corresponds to one specific example of a “first layer” of the present disclosure, and the second layer 23B corresponds to one specific example of a “second layer” of the present disclosure.

### (1-1. Configuration of Imaging Element)

For example, the imaging element 10 is of what is called a longitudinal spectral type, in which one photoelectric conversion section 20 and two photoelectric conversion regions 32B and 32R are stacked in a longitudinal direction. The photoelectric conversion section 20 is provided on a back surface (first surface 30A) side of the semiconductor substrate 30. The photoelectric conversion regions 32B and 32R are formed embedded in the semiconductor substrate 30 and are stacked in a thickness direction of the semiconductor substrate 30.

The photoelectric conversion section 20 and the photoelectric conversion regions 32B and 32R selectively detect light having wavelength ranges different from each other and perform photoelectric conversion. For example, the photoelectric conversion section 20 acquires a green (G) signal. The photoelectric conversion regions 32B and 32R acquire blue (B) and red (R) signals, respectively, depending on a difference in absorption coefficient. This allows the imaging element 10 to acquire a plurality of types of color signals in one pixel without using a color filter.

It is to be noted that the present embodiment describes a case where an electron is read out as a signal charge from an electron-hole pair (exciton) generated by photoelectric conversion (a case where an n-type semiconductor region is used as a photoelectric conversion layer). In addition, “+ (plus)” appended to “p” or “n” in the figure indicates a high concentration of a p-type or n-type impurity.

For example, on a surface (second surface 30B) of the semiconductor substrate 30, a floating diffusion (floating diffusion layer) FD1 (region 36B in the semiconductor substrate 30), an FD2 (region 37C in the semiconductor substrate 30), an FD3 (region 38C in the semiconductor substrate 30), transfer transistors Tr2 and Tr3, an amplifier transistor (modulation element) AMP, a reset transistor RST, and a selection transistor SEL are provided. On the second surface 30B of the semiconductor substrate 30, a multilayer wiring layer 40 is further provided via a gate insulating layer 33. For example, the multilayer wiring layer 40 has a configuration in which wiring layers 41, 42, and 43 are stacked within an insulating layer 44. In a periphery around the semiconductor substrate 30, namely, the peripheral region 1B around the pixel section 1A, a vertical drive circuit 111, a column signal processing circuit 112, a horizontal drive circuit 113, an output circuit 114, a control circuit 115, and an input/output terminal 116 or the like as described later are provided.

It is to be noted that in the drawing, the first surface 30A side of the semiconductor substrate 30 is illustrated as a light incoming side S1, and the second surface 30B side as a wiring layer side S2.

In the photoelectric conversion section 20, between the lower electrode 21 and the upper electrode 26 disposed opposed to each other, a semiconductor layer 23, a protective layer 24, and a photoelectric conversion layer 25 formed by using an organic material are stacked in this order from the lower electrode 21 side. In the semiconductor layer 23, as described above, the first layer 23A and the second layer 23B having a difference in at least one of material composition, crystallinity, impurity concentration contained, or constituent element are provided above the readout electrode 21A and the storage electrode 21B, respectively. The photoelectric conversion layer 25 includes a p-type semiconductor and an n-type semiconductor and has a bulk heterojunction structure within the layer. The bulk heterojunction structure includes a p/n junction surface formed as a result of mixing the p-type semiconductor and the n-type semiconductor.

The photoelectric conversion section 20 further includes the insulating layer 22 between the lower electrode 21 and the semiconductor layer 23. For example, the insulating layer 22 is provided over an entire surface of the pixel section 1A and also has the opening 22H on the readout electrode 21A included in the lower electrode 21. The readout electrode 21A is electrically coupled to the semiconductor layer 23 via this opening 22H. The first layer 23A in the semiconductor layer 23 is provided above the storage electrode 21B via the insulating layer 22. The second layer 23B in the semiconductor layer 23 is provided above the readout electrode 21A, for example, along the opening 22H from a side surface to a bottom surface thereof, and is electrically coupled to the readout electrode 21A at the bottom surface.

It is to be noted that although FIG. 1 illustrates an example in which the semiconductor layer 23, the protective layer 24, the photoelectric conversion layer 25, and the upper electrode 26 are formed separately for each imaging element 10, the semiconductor layer 23, the protective layer 24, the photoelectric conversion layer 25, and the upper electrode 26 may be provided as, for example, a continuous layer shared by a plurality of imaging elements 10.

Between the first surface 30A of the semiconductor substrate 30 and the lower electrode 21, for example, an insulating layer 27 and an interlayer insulating layer 28 are stacked. In the insulating layer 27, a layer having a fixed charge (fixed charge layer) 27A and a dielectric layer 27B having insulating property are stacked in this order from the semiconductor substrate 30 side.

The photoelectric conversion regions 32B and 32R enable a longitudinal dispersion of light by utilizing a difference in wavelength of light absorbed in accordance with a depth of incoming light in the semiconductor substrate 30 including a silicon substrate, and each have a pn junction in a predetermined region of the semiconductor substrate 30.

Between the first surface 30A and the second surface 30B of the semiconductor substrate 30, a through electrode 34 is provided. The through electrode 34 is electrically coupled to the readout electrode 21A, and via the through electrode 34, for example, the photoelectric conversion section 20 is coupled to a gate Gamp of an amplifier transistor AMP and to one source/drain region 36B of the reset transistor RST (reset transistor Tr1rst) that also serves as the floating diffusion FD1. This allows the imaging element 10 to transfer a carrier (here, electron) generated in the photoelectric conversion section 20 provided on the first surface 30A side of the semiconductor substrate 30 to the second surface 30B side of the semiconductor substrate 30 in a satisfactory manner via the through electrode 34, thereby improving a characteristic.

The through electrode 34 has a lower end coupled to a wiring (coupling section 41A) in a wiring layer 41, and the coupling section 41A is coupled to the gate Gamp of the amplifier transistor AMP via a lower first contact 45. The coupling section 41A is coupled to the floating diffusion FD1 (region 36B) via, for example, a lower second contact 46. The through electrode 34 has an upper end coupled to the readout electrode 21A via, for example, a pad 39A and an upper first contact 39D.

A protective layer 51 is provided above the photoelectric conversion section 20. In the protective layer 51, for example, a wiring 52 is provided to electrically couple the upper electrode 26 and the peripheral circuit section 130 around the pixel section 1A. Above the protective layer 51, an optical member, such as a planarization layer (not illustrated) or an on-chip lens 54, is further provided.

In the imaging element 10 of the present embodiment, incoming light to the photoelectric conversion section 20 from the light incoming side S1 is absorbed by the photoelectric conversion layer 25. An exciton generated thereby moves to an interface between an electron donor and an electron acceptor included in the photoelectric conversion layer 25 and undergoes exciton separation, that is, dissociates into an electron and a hole. Each carrier (the electron and hole) generated here is transported to a different electrode by diffusion due to a difference in carrier concentration or by an internal electric field due to a difference in work function between an anode (for example, the upper electrode 26) and a cathode (for example, the lower electrode 21), to be detected as a photoelectric current. In addition, it is also possible to control a transport direction of the electron and hole by applying an electric potential between the lower electrode 21 and the upper electrodes 26.

The following will describe the configuration, material, etc. of each section in detail.

The photoelectric conversion section 20 is an organic photoelectric conversion element that generates an exciton by absorbing, for example, green light corresponding to all or a part of a selective wavelength range (for example, 450 nm or more to 650 nm or less).

For example, the lower electrode 21 includes the readout electrode 21A, the storage electrode 21B, and the transfer electrode 21C. The readout electrode 21A, the storage electrode 21B, and the transfer electrode 21C are stacked in the insulating layer 22, for example, in an order of the readout electrode 21A, the storage electrode 21B, and the transfer electrode 21C from the semiconductor substrate 30 side in a cross section. Specifically, the readout electrode 21A, the storage electrode 21B, and the transfer electrode 21C are each provided per unit pixel P. As illustrated in FIG. 2, for example, the readout electrode 21A is provided at one of four corners of the unit pixel P. The storage electrode 21B is provided around the readout electrode 21A, for example, in an upper layer than the readout electrode 21A. Although not illustrated in FIG. 2, the transfer electrode 21C is provided to surround the readout electrode 21A, for example, between the readout electrode 21A and the storage electrode 21B, both in plan view and in cross-sectional view.

The readout electrode 21A is intended to transfer the carrier generated in the photoelectric conversion layer 25 to the floating diffusion FD1. The readout electrode 21A is coupled to the floating diffusion FD1 via, for example, the upper first contact 39D, the pad 39A, the through electrode 34, the coupling section 41A, and the lower second contact 46.

For example, the storage electrode 21B is intended to accumulate an electron as a signal charge in the semiconductor layer 23, from the carrier generated in the photoelectric conversion layer 25. The storage electrode 21B is provided per unit pixel P, directly opposed to light receiving surfaces of the photoelectric conversion regions 32B and 32R formed in the semiconductor substrate 30, in a region covering these light receiving surfaces. It is preferable for the storage electrode 21B to be larger than the readout electrode 21A, thereby allowing an accumulation of a large number of carriers. Independently of the readout electrode 21A, a voltage is to be applied to the storage electrode 21B via, for example, an upper sixth contact 39L, a pad 39K, an upper fifth contact 39J, a pad 39H, an upper third contact 39F, and a pad 39C.

The transfer electrode 21C is intended to improve a transfer efficiency of the carrier accumulated above the storage electrode 21B to the readout electrode 21A. Independently of the readout electrode 21A and the storage electrode 21B, a voltage is to be applied to the transfer electrode 21C via, for example, an upper fourth contact 39I, a pad 39G, an upper second contact 39E, and a pad 39B.

Although not illustrated in FIG. 1, a shield electrode 21D to partition each unit pixel P is further provided around the storage electrode 21B. The shield electrode 21D is intended to prevent capacitive coupling between adjacent unit pixels P, and, for example, a fixed potential is applied thereto.

The lower electrode 21 includes a conductive film having a light transmissivity, which is, for example, ITO (indium tin oxide). As a constituent material for the lower electrode 21, other than ITO, a tin oxide (SnO₂)-based material with a dopant added or a zinc oxide-based material resulting from an addition of a dopant to a zinc oxide (ZnO) may be used. As the zinc oxide-based material, for example, it is possible to name aluminum zinc oxide (AZO) with aluminum (Al) added as a dopant, gallium zinc oxide (GZO) with gallium (Ga) added, or indium zinc oxide (IZO) with indium (In) added. In addition, other than this, IGZO, ITZO, CuI, InSbO₄, ZnMgO, CuInO₂, MgIN₂O₄, CdO, ZnSnO₃ or the like may also be used.

The insulating layer 22 is intended to electrically separate the storage electrode 21B and the semiconductor layer 23. The insulating layer 22 is provided, for example, on the interlayer insulating layer 28 to cover the lower electrode 21. As described above, the insulating layer 22 has the opening 22H on the readout electrode 21A in the lower electrode 21, and the readout electrode 21A and the semiconductor layer 23 are electrically coupled to each other via this opening 22H. The insulating layer 22 includes, for example, a monolayer film including one type or a multilayer film including two or more types selected from silicon oxide (SiOₓ), hafnium oxide (HfOₓ), aluminum oxide (AlOₓ), silicon nitride (SiNₓ), and silicon oxynitride (SiON) or the like. The insulating layer 22 has a thickness of, for example, 20 nm to 500 nm.

The semiconductor layer 23 is intended to accumulate the carrier generated in the photoelectric conversion layer 25. As described above, the semiconductor layer 23 is provided between the lower electrode 21 and the photoelectric conversion layer 25. Specifically, the semiconductor layer 23 is provided on the insulating layer 22 as well as over a side surface and a bottom surface of the opening 22H formed on the readout electrode 21A, and is electrically coupled to the readout electrode 21A in the bottom surface of the opening 22H. The semiconductor layer 23 includes the first layer 23A and the second layer 23B having a difference. The first layer 23A and the second layer 23B are provided above the storage electrode 21B and the readout electrode 21A, respectively, and the second layer 23B is electrically coupled to the readout electrode 21A via the opening 22H. The following will describe the difference between the first layer 23A and the second layer 23B.

For example, it is preferable that the first layer 23A, which is provided above the storage electrode 21B and in which the carrier (in this case, electron) generated in the photoelectric conversion layer 25 is accumulated, have fewer defects that trap a carrier. For the second layer 23B, which is provided above the readout electrode 21A and provided along the side surface and the bottom surface of the opening 22H of the insulating layer 22, it is important to have embeddability.

As described above, for the semiconductor layer 23, it is possible to achieve both low trapping above the storage electrode 21B and embeddability in the opening 22H by causing the first layer 23A and the second layer 23B to have a difference in at least one of material composition, crystallinity, impurity concentration contained, or constituent element.

For example, the first layer 23A and the second layer 23B have a crystalline property different from each other. Specifically, for example, the first layer 23A has crystallinity and the second layer 23B has amorphousness. For example, an impurity of different concentration is added to each of the first layer 23A and the second layer 23B, and for example, it is preferable that the impurity concentration of the first layer 23A be lower than that of the second layer 23B, for example, one tenth or less. Here, the impurity contained in the first layer 23A and the second layer 23B is, for example, carbon (C).

It is possible to form the semiconductor layer 23, using, for example, an oxide semiconductor material including at least one type of element selected from indium (In), gallium (Ga), silicon (Si), zinc (Zn), aluminum (Al), and tin (Sn). In the present embodiment, an electron in the carrier generated in the photoelectric conversion layer 25 is used as a signal charge. This makes it possible to form the semiconductor layer 23, using an n-type oxide semiconductor material. Specifically, as a constituent material for the semiconductor layer 23, it is possible to name IGZO, Ga₂O₃, GZO, IZO, ITO, InGaAlO, InGaSiO or the like. For example, in a case where the first layer 23A is formed using the above oxide semiconductor material, it is preferable that an amount of In contained in the first layer 23A be larger than the amount of In contained in the second layer 23B, and it is preferable that an amount of Ga contained in the first layer 23A be smaller than the amount of Ga contained in the second layer 23B. In addition, among the oxide semiconductor materials described above, it is preferable that the second layer 23B contain Al.

It is possible to form the first layer 23A, using, for example, a physical vapor deposition (PVD) method. It is possible to form the second layer 23B, using, for example, an atomic layer deposition (ALD) method. The semiconductor layer 23 has a thickness of, for example, 10 nm to 300 nm.

The protective layer 24 is intended to prevent desorption of oxygen from the oxide semiconductor material included in the semiconductor layer 23. For example, it is possible to form the protective layer 24, using a metal oxide containing at least one type of element selected from tantalum (Ta), titanium (Ti), vanadium (V), niobium (Nb), tungsten (W), zirconium (Zr), hafnium (Hf), scandium (Sc), yttrium (Y), lanthanum (La), gallium (Ga), and magnesium (Mg). Specifically, as a constituent material for the protective layer 24, for example, it is possible to name tantalum oxide (Ta₂O₅), titanium oxide (TiO₂), vanadium oxide (V₂O₅), niobium oxide (Nb₂O₅), tantalum oxide (W₂O₃), zirconium oxide (ZrO₂), hafnium oxide (HfO₂), scandium oxide (Sc₂O₃), yttrium oxide (Y₂O₃), lanthanum oxide (La₂O₃), gallium oxide (Ga₂O₃), and magnesium oxide (MgO). For example, the protective layer 24 having a thickness of one atomic layer is effective, and it is preferable that the protective layer 24 have a thickness of, for example, 0.5 nm or more and 10 nm or less.

As illustrated in FIG. 3A, the protective film 29 may be embedded between the second layer 23B provided above the readout electrode 21A and along the side surface and the bottom surface of the opening 22H, and the protective layer 24. The protective film 29 is intended to prevent oxygen desorption and includes, for example, an insulating film such as SiOₓ or AlOₓ.

It is to be noted that it is not always necessary to provide the second layer 23B along the side surface and the bottom surface of the opening 22H, and as illustrated in FIG. 3B, for example, the second layer 23B may be provided to fill the opening 22H. In that case, the protective film 29 is omitted.

The photoelectric conversion layer 25 converts light energy into electrical energy. The photoelectric conversion layer 25 includes, for example, two or more types of organic materials (a p-type semiconductor material or an n-type semiconductor material) each of which functions as a p-type semiconductor or an n-type semiconductor. The photoelectric conversion layer 25 has a junction surface (p/n junction surface) between the p-type semiconductor material and the n-type semiconductor material within the layer. A p-type semiconductor functions relatively as an electron donor (donor), and an n-type semiconductor functions relatively as an electron acceptor (acceptor). The photoelectric conversion layer 25 provides a field in which an exciton generated when light is absorbed is separated into an electron and a hole. Specifically, the exciton is separated into an electron and a hole at an interface between the electron donor and the electron acceptor (p/n junction surface).

Other than the p-type semiconductor material and the n-type semiconductor material, the photoelectric conversion layer 25 may also include what is called a dye material, which is an organic material that photoelectrically converts light having a predetermined wavelength range while transmitting light having another wavelength range. In a case where the photoelectric conversion layer 25 is formed using three types of organic materials, that is, the p-type semiconductor material, the n-type semiconductor material, and the dye material, it is preferable that the p-type semiconductor material and the n-type semiconductor material have light transmissivity in a visible region (for example, 450 nm to 800 nm). The photoelectric conversion layer 25 has a thickness of, for example, 50 nm to 500 nm.

As the organic material included in the photoelectric conversion layer 25, for example, it is possible to name a quinacridone derivative, a naphthalene derivative, an anthracene derivative, a phenanthrene derivative, a tetracene derivative, a pyrene derivative, a perylene derivative, and a fluoranthene derivative. The photoelectric conversion layer 25 includes a combination of two or more types of the organic materials described above. The organic materials described above function as a p-type semiconductor or an n-type semiconductor depending on the combination.

It is to be noted that the organic material included in the photoelectric conversion layer 25 is not particularly limited. For example, other than the organic materials described above, it is possible to use a polymer of phenylenevinylene, fluorene, carbazole, indole, pyrene, pyrrole, picoline, thiophene, acetylene and diacetylene or the like, or a derivative thereof. Alternatively, it is possible to use a metal complex dye, a cyanine dye, a merocyanine dye, a phenylxanthene dye, a triphenylmethane dye, a rhodacyanine dye, a xanthene dye, a macrocyclic azaazulene dye, an azulene dye, a naphthoquinone dye, an anthraquinone dye, a fused polycycle aromatic compound such as pyrene, a chain compound with fused aromatic or heterocyclic compounds, quinoline having a squarylium group and a croconitumetin group as a bonding chain, two nitrogen-containing heterocycles such as benzothiazole and benzoxazole, or a cyanine-like dye bonded by a squarylium group and a croconitumetin group, or the like. It is to be noted that as the metal complex dye, it is possible to name a dithiol metal complex dye, a metal phthalocyanine dye, a metal porphyrin dye, or a ruthenium complex dye. Of these, the ruthenium complex dye is particularly preferable, but the material is not limited to the above.

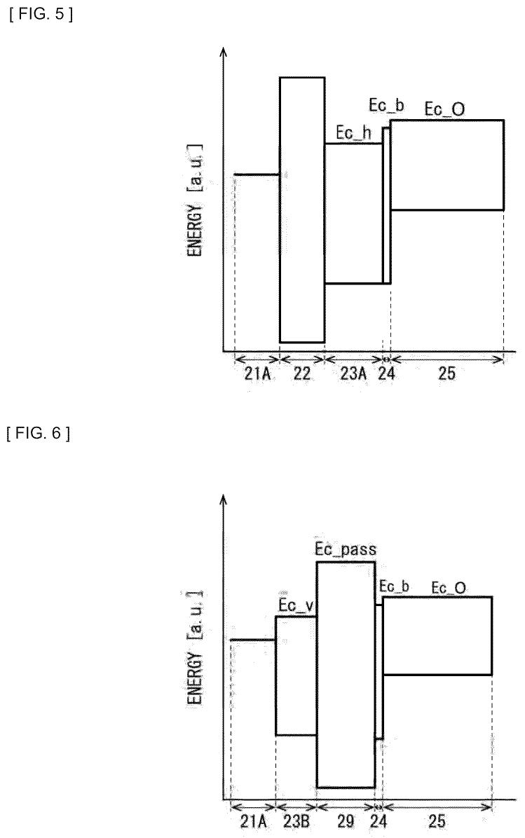

FIG. 4 illustrates an energy level of the readout electrode 21A, the second layer 23B, and the first layer 23A corresponding to a line A-A illustrated in FIG. 3A. FIG. 5 illustrates an energy level of the storage electrode 21B, the insulating layer 22, the first layer 23A, the protective layer 24, and the photoelectric conversion layer 25 corresponding to a line B-B illustrated in FIG. 3A. FIG. 6 illustrates an energy level of the readout electrode 21A, the second layer 23B, the protective film 29, the protective layer 24, and the photoelectric conversion layer 25 corresponding to a line C-C illustrated in FIG. 3A. It is to be noted that a direction away from a vacuum level is defined to indicate a larger positive energy.

For example, it is preferable that an energy level (Ec_v) at a lowest end of a conduction band of the second layer 23B be equal to an energy level (Ec_h) at a lowest end of a conduction band of the first layer 23A or be deeper than the energy level (Ec_h) at the lowest end of the conduction band of the first layer 23A (Ec_v≥Ec_h). It is preferable that the energy level (Ec_h) at the lowest end of the conduction band of the first layer 23A be equal to an energy level (Ec_b) at a lowest end of a conduction band of the protective layer 24 or be deeper than the energy level (Ec_b) at the lowest end of the conduction band of the protective layer 24, and that the energy level (Ec_b) at the lowest end of the conduction band of the protective layer 24 be equal to a LUMO (Ec_O) of the photoelectric conversion layer or be deeper than the energy level (Ec_O) at a lowest end of a conduction band of the photoelectric conversion layer (Ec_h≥Ec_b≥Ec_O). It is preferable that the energy level at the lowest end of the conduction band of the second layer 23B (Ec_v) be shallower than an energy level (Ec_pas) at a lowest end of a conduction band of the protective film 29, that the energy level (Ec_b) at the lowest end of the conduction band of the protective film 29 be shallower than the LUMO (Ec_O) of the photoelectric conversion layer, and that the LUMO (Ec_O) of the photoelectric conversion layer be shallower than the energy level (EC_b) at the lowest end of the conduction band of the protective layer 24 (Ec_v>Ec_pas, Ec_pas<Ec_O<Ec_b).

The upper electrode 26 includes a conductive film having light transmissivity as with the upper electrode 26 and includes, for example, ITO (indium tin oxide). Other than this ITO, a tin oxide (SnO₂)-based material with a dopant added or a zinc oxide-based material resulting from an addition of a dopant to a zinc oxide (ZnO) may be used as a constituent material for the upper electrode 26. As the zinc oxide-based material, for example, it is possible to name an aluminum zinc oxide (AZO) with aluminum (Al) added as a dopant, a gallium zinc oxide (GZO) with gallium (Ga) added, and an indium zinc oxide (IZO) with indium (In) added. In addition, other than this, IGZO, ITZO, CuI, InSbO₄, ZnMgO, CuInO₂, MgIN₂O₄, CdO, ZnSnO₃, or the like may also be used. The upper electrode 26 may be separated for each pixel or may be formed as a common electrode for each pixel. The upper electrode 26 has a thickness of, for example, 10 nm to 200 nm.

It is to be noted that the photoelectric conversion section 20 may include another layer between the lower electrode 21 and the photoelectric conversion layer 25 (for example, between the protective layer 24 and the photoelectric conversion layer 25) and between the photoelectric conversion layer 25 and the upper electrode 26. For example, in the photoelectric conversion section 20, the protective layer 24, a buffer layer that also serves as an electron blocking film, the photoelectric conversion layer 25, a buffer layer that also serves as a hole blocking film, and a work function adjustment layer or the like may be stacked in order from the lower electrode 21 side. In addition, for example, the photoelectric conversion layer 25 may have a pin bulk-heterojunction configuration in which a p-type blocking layer, a layer including a p-type semiconductor and an n-type semiconductor (i-layer), and an n-type blocking layer are stacked.

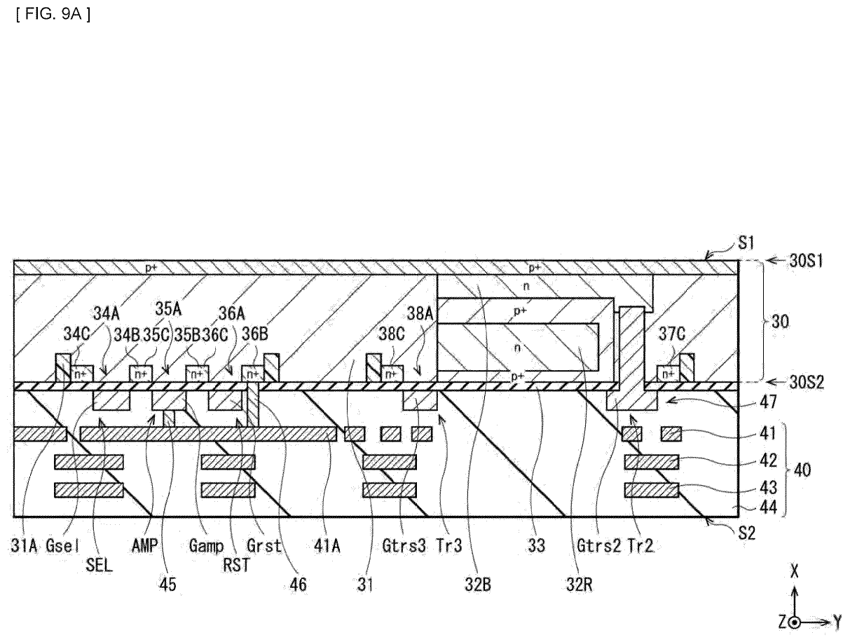

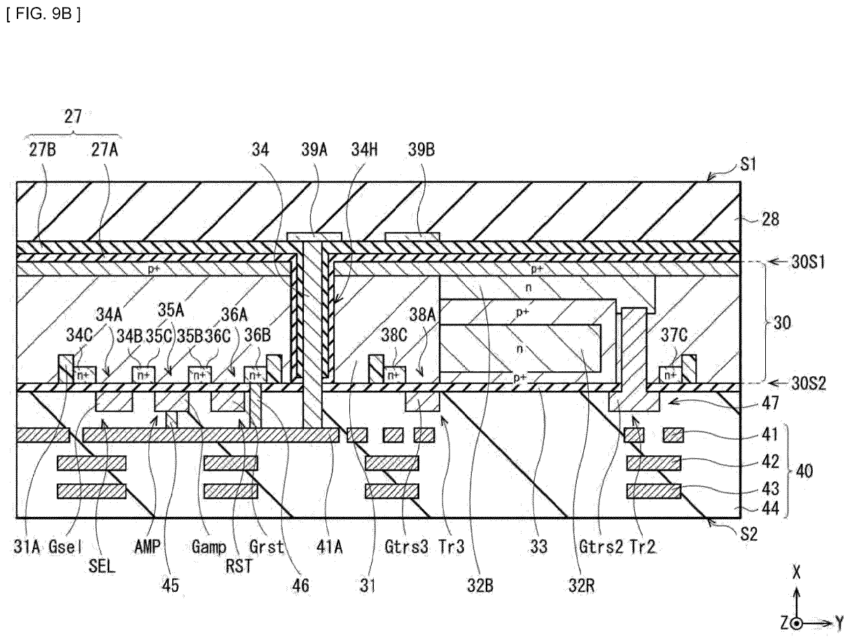

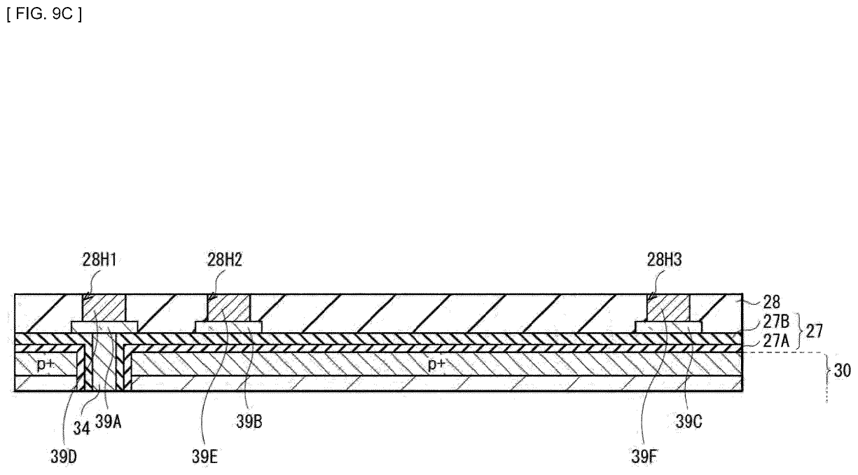

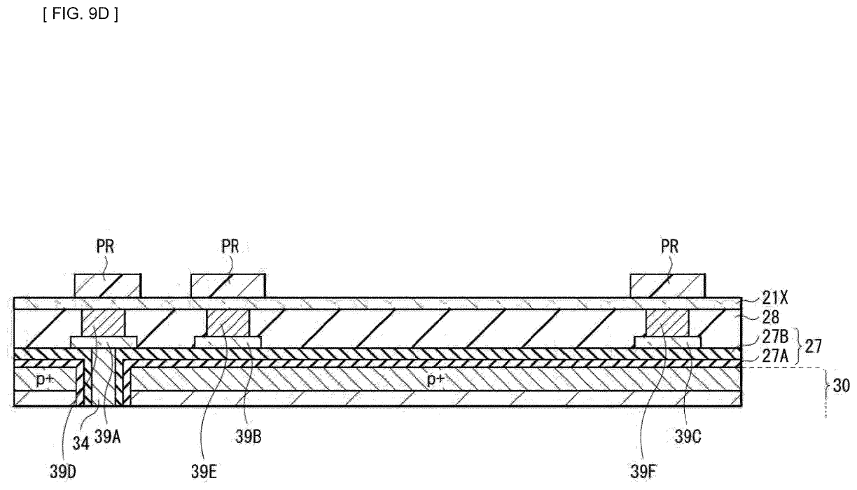

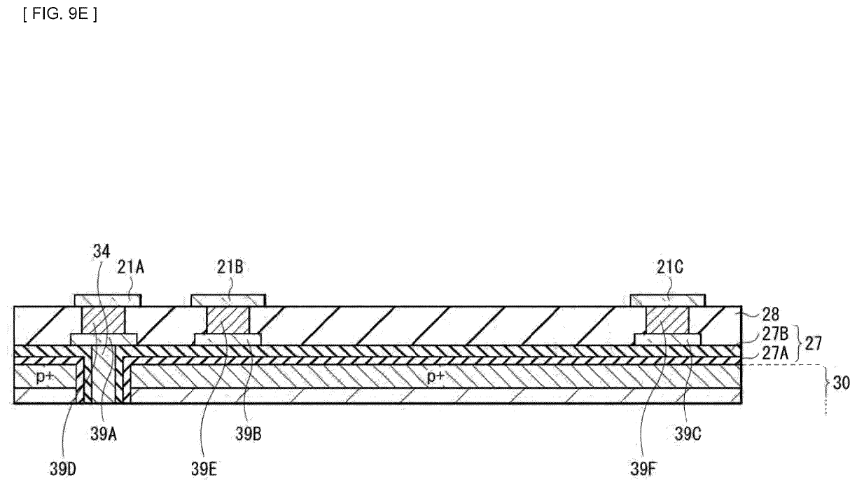

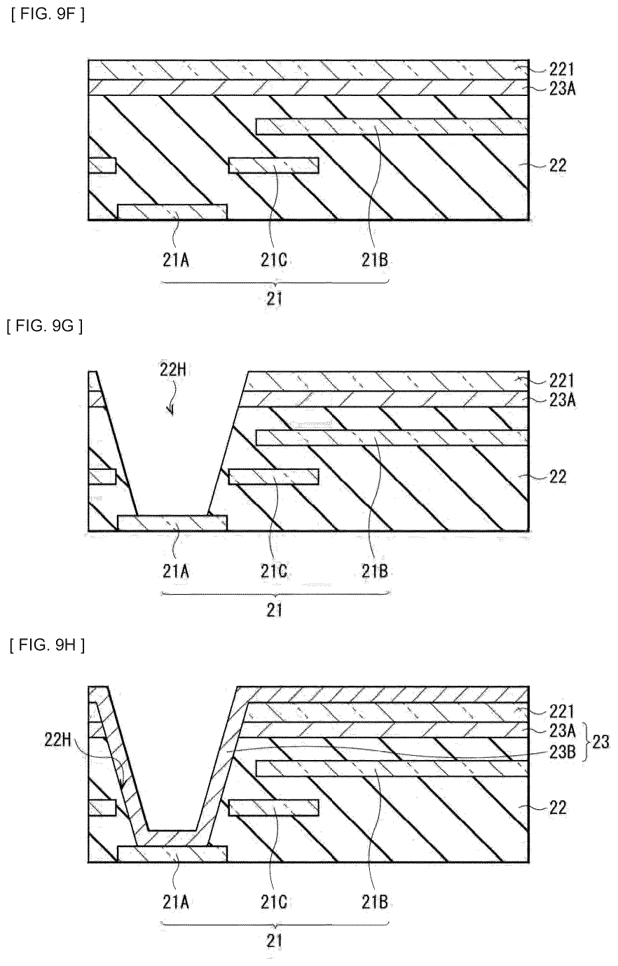

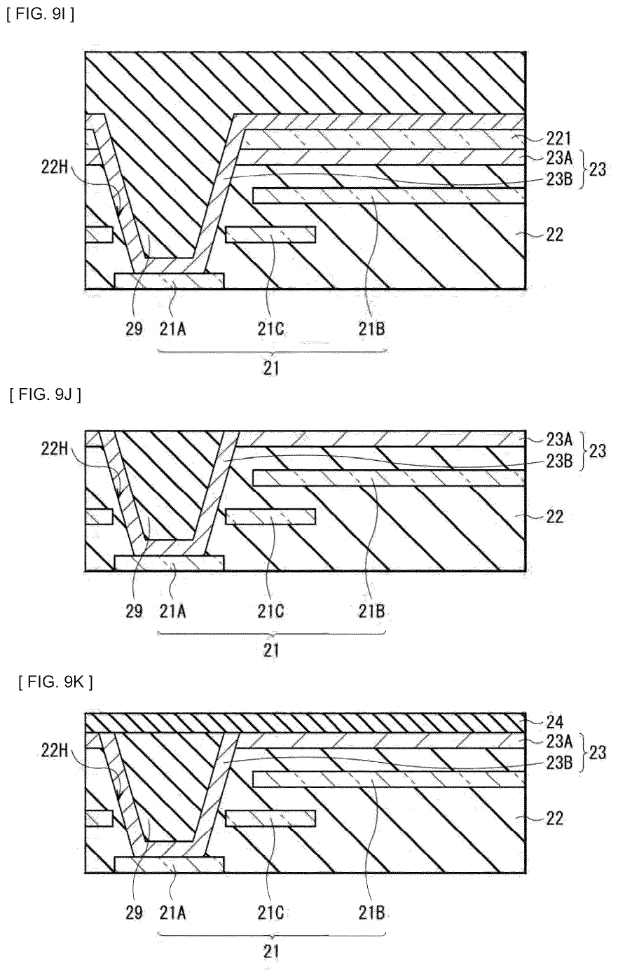

The insulating layer 27 covers the first surface 30A of the semiconductor substrate 30 to reduce an interface level with the semiconductor substrate 30 and to suppress a generation of dark current from the interface with the semiconductor substrate 30. In addition, the insulating layer 27 extends from the first surface 30A of the semiconductor substrate 30 to a side surface of an opening 34H (see FIG. 9B) in which the through electrode 34 penetrating through the semiconductor substrate 30 is formed. For example, the insulating layer 27 has a stacked configuration of the fixed charge layer 27A and the dielectric layer 27B.

The fixed charge layer 27A may be a film having a positive fixed charge or may be a film having a negative fixed charge. As a constituent material for the fixed charge layer 27A, it is preferable to form the fixed charge layer 27A using a semiconductor material or a conductive material having a wider band gap than that of the semiconductor substrate 30. This makes it possible to suppress the generation of dark current at the interface of the semiconductor substrate 30. For example, as the constituent material for the fixed charge layer 27A, it is possible to name hafnium oxide (HfOx), aluminum oxide (AlOₓ), zirconium oxide (ZrOₓ), tantalum oxide (TaOₓ), titanium oxide (TiOₓ), lanthanum oxide (LaOₓ), praseodymium oxide (PrOₓ), cerium oxide (CeOₓ), neodymium oxide (NdOₓ), promethium oxide (PmOₓ), samarium oxide (SmOₓ), europium oxide (EuOₓ), gadolinium oxide (GdOₓ), terbium oxide (TbOₓ), dysprosium oxide (DyOₓ), holmium oxide (HoOₓ), thulium oxide (TmOₓ), ytterbium oxide (YbOₓ), lutetium oxide (LuOₓ), yttrium oxide (YOₓ), hafnium nitride (HfNₓ), aluminum nitride (AlNₓ), hafnium oxynitride (HfOₓN_{y}), and aluminum oxynitride (AlOₓN_{y}) or the like.

The dielectric layer 27B is intended to prevent reflection of light caused by a difference in refractive index between the semiconductor substrate 30 and the interlayer insulating layer 28. As a constituent material for the dielectric layer 27B, it is preferable to use a material having a refractive index between the refractive index of the semiconductor substrate 30 and the refractive index of the interlayer insulating layer 28. For example, as the constituent material for the dielectric layer 27B, it is possible to name silicon oxide, TEOS, silicon nitride, and silicon oxynitride (SiON) or the like.

For example, the interlayer insulating layer 28 includes a monolayer film including one type selected from silicon oxide, silicon nitride, and silicon oxynitride or the like, or a multilayer film including two or more types of these.

For example, the semiconductor substrate 30 includes an n-type silicon (Si) substrate and has a p-well 31 in a predetermined region.

The photoelectric conversion regions 32B and 32R each include a photodiode (PD) having a pn junction in a predetermined region of the semiconductor substrate 30, thereby enabling a longitudinal dispersion of light by utilizing the fact that a wavelength of light absorbed in the Si substrate varies depending on the depth of the incoming light. For example, the photoelectric conversion region 32B selectively detects blue light and accumulates a signal charge corresponding to blue, and is disposed at a depth that enables efficient photoelectric conversion of blue light. For example, the photoelectric conversion region 32R selectively detects red light and accumulates a signal charge corresponding to red, and is disposed at a depth that enables efficient photoelectric conversion of red light. It is to be noted that blue (B) and red (R) are colors corresponding to a wavelength range of 450 nm to 495 nm and a wavelength range of 620 nm to 750 nm, respectively. It is sufficient to allow each of the photoelectric conversion regions 32B and 32R to detect light in all or a part of a corresponding one of the wavelength ranges.

The photoelectric conversion region 32B includes, for example, a p+ region that is to be a hole accumulation layer and an n region that is to be an electron accumulation layer. The photoelectric conversion region 32R, for example, includes a p+ region that is to be a hole accumulation layer and an n region that is to be an electron accumulation layer (having a p-n-p laminate configuration). The n region in the photoelectric conversion region 32B is coupled to a transfer transistor Tr2 of a vertical type. The p+ region in the photoelectric conversion region 32B is bent along the transfer transistor Tr2, leading to the p+ region of the photoelectric conversion region 32R.

For example, the gate insulating layer 33 includes a monolayer film including one type selected from silicon oxide, silicon nitride, and silicon oxynitride or the like, or a multilayer film including two or more types of these.

The through electrode 34 is provided between the first surface 30A and the second surface 30B of the semiconductor substrate 30 and functions as a connector to couple the photoelectric conversion section 20 to the gate Gamp of the amplifier transistor AMP and the floating diffusion FD1, while serving as a transmission path for the carrier generated in the photoelectric conversion section 20. Next to the floating diffusion FD1 (one source/drain region 36B of the reset transistor RST), a reset gate Grst of the reset transistor RST is disposed. This allows the reset transistor RST to reset the carrier accumulated in the floating diffusion FD1.

The pads 39A and 39B, the upper first contact 39D, the upper second contact 39E, the lower first contact 45, the lower second contact 46, and the wiring 52 are formed using, for example, a doped silicon material such as PDAS (Phosphorus Doped Amorphous Silicon), or a metallic material such as aluminum (Al), tungsten (W), titanium (Ti), cobalt (Co), hafnium (Hf), or tantalum (Ta).

The protective layer 51 and the on-chip lens 54 include a material having light transmissivity, including, for example, a monolayer film including any one of silicon oxide, silicon nitride, and silicon oxynitride or the like, or a stacked film including two or more types of these. This protective layer 51 has a thickness of, for example, 100 nm to 30000 nm.

For example, a light shielding film 53 may be provided within the protective layer 51. For example, the light shielding film 53, together with the wiring 52, is provided in the protective layer 51 to cover a region of the readout electrode 21A which is in direct contact with the semiconductor layer 23, but not over at least the storage electrode 21B. It is possible to form the light shielding film 53 using, for example, tungsten (W), aluminum (Al), an alloy of Al and copper (Cu) or the like.

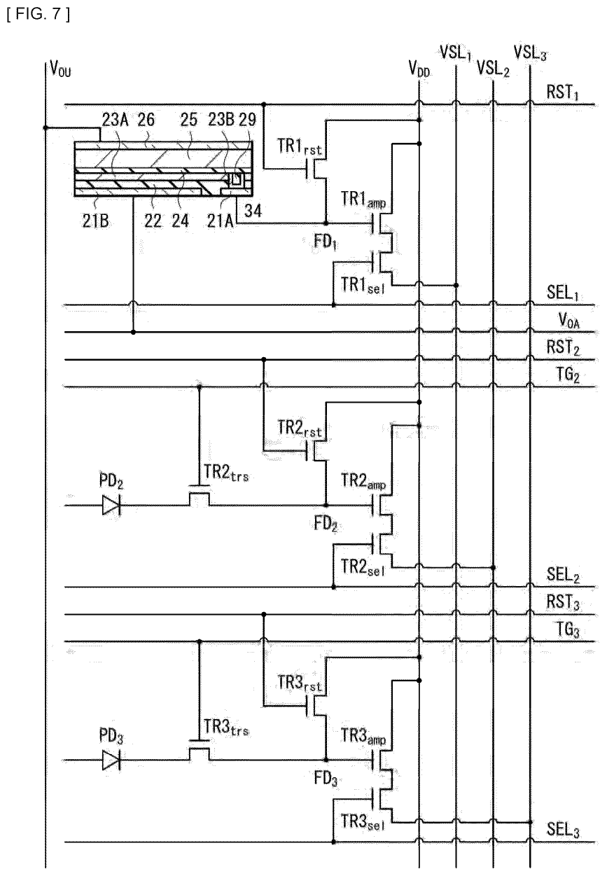

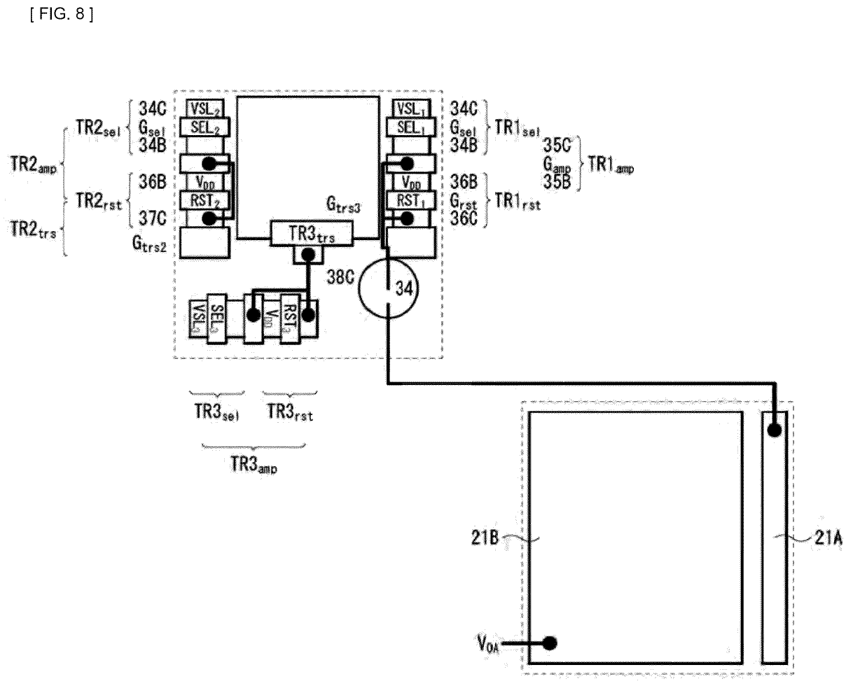

FIG. 7 is an equivalent circuit diagram of the imaging element 10 illustrated in FIG. 1. FIG. 8 schematically illustrates an arrangement of each transistor included in the lower electrode 21 and a control section of the imaging element 10 illustrated in FIG. 1.

The reset transistor RST (reset transistor TR1rst) is intended to reset the carrier transferred from the photoelectric conversion section 20 to the floating diffusion FD1, and includes, for example, a MOS transistor. Specifically, the reset transistor TR1rst includes the reset gate Grst, a channel formation region 36A, and the source/drain regions 36B and 36C. The reset gate Grst is coupled to a reset line RST1, and one source/drain region 36B of the reset transistor TR1rst also serves as the floating diffusion FD1. Another source/drain region 36C included in the reset transistor TR1rst is coupled to a power line VDD.

The amplifier transistor AMP (amplifier transistor TR1amp) is a modulation element that modulates an amount of the electric charge generated in photoelectric conversion section 20 to a voltage, and includes, for example, a MOS transistor. Specifically, the amplifier transistor AMP includes a gate Gamp, a channel formation region 35A, and source/drain regions 35B and 35C. The gate Gamp is coupled to the readout electrode 21A and one source/drain region 36B (floating diffusion FD1) of the reset transistor TR1rst via the lower first contact 45, the coupling section 41A, the lower second contact 46, and the through electrode 34 or the like. In addition, one source/drain region 35B shares a region with another source/drain region 36C included in the reset transistor TR1rst, and is coupled to the power line VDD.

The selection transistor SEL (selection transistor TR1sel) includes a gate Gsel, a channel formation region 34A, and source/drain regions 34B and 34C. The gate Gsel is coupled to the selection line SEL1. One source/drain region 34B shares a region with another source/drain region 35C included in the amplifier transistor AMP, and another source/drain region 34C is coupled to the signal line (data output line) VSL1.

The transfer transistor TR2 (transfer transistor TR2trs) is intended to transfer the signal charge corresponding to blue, which is generated and accumulated in the photoelectric conversion region 32B, to the floating diffusion FD2. Because the photoelectric conversion region 32B is formed at a deep position from the second surface 30B of the semiconductor substrate 30, it is preferable that the transfer transistor TR2trs in the photoelectric conversion region 32B include a transistor of a vertical type. The transfer transistor TR2trs is coupled to a transfer gate line TG2. In the region 37C near a gate Gtrs2 of the transfer transistor TR2trs, the floating diffusion FD2 is provided. The carrier accumulated in the photoelectric conversion region 32B is read out to the floating diffusion FD2 via a transfer channel formed along the gate Gtrs2.

A transfer transistor TR3 (transfer transistor TR3trs) is intended to transfer the signal charge corresponding to red, which is generated and accumulated in the photoelectric conversion region 32R, to the floating diffusion FD3 and includes, for example, a MOS transistor. The transfer transistor TR3trs is coupled to a transfer gate line TG3. A floating diffusion FD3 is provided in the region 38C near a gate Gtrs3 of the transfer transistor TR3trs. The carrier accumulated in the photoelectric conversion region 32R is read out to the floating diffusion FD3 via the transfer channel formed along the gate Gtrs3.

On the second surface 30B side of the semiconductor substrate 30, a reset transistor TR2rst, an amplifier transistor TR2amp, and a selection transistor TR2sel are further provided to be included in the control section of the photoelectric conversion region 32B. Furthermore, a reset transistor TR3rst, an amplifier transistor TR3amp, and a selection transistor TR3sel are provided to be included in the control section of the photoelectric conversion region 32R.

The reset transistor TR2rst includes a gate, a channel formation region, and a source/drain region. The reset transistor TR2rst has a gate coupled to the reset line RST2, and one source/drain region of the reset transistor TR2rst is coupled to the power line VDD. Another source/drain region of the reset transistor TR2rst also serves as the floating diffusion FD2.

The amplifier transistor TR2amp includes a gate, a channel formation region, and a source/drain region. The gate is coupled to another source/drain region (floating diffusion FD2) of the reset transistor TR2rst. One source/drain region included in the amplifier transistor TR2amp shares a region with one source/drain region included in the reset transistor TR2rst, and is coupled to the power line VDD.

The selection transistor TR2sel includes a gate, a channel formation region, and a source/drain region. The gate is coupled to a selection line SEL2. One source/drain region included in the selection transistor TR2sel shares a region with another source/drain region included in the amplifier transistor TR2amp. Another source/drain region included in the selection transistor TR2sel is coupled to a signal line (data output line) VSL2.

The reset transistor TR3rst includes a gate, a channel formation region, and a source/drain region. The reset transistor TR3rst has a gate coupled to a reset line RST3, and one source/drain region included in the reset transistor TR3rst is coupled to the power line VDD. Another source/drain region included in the reset transistor TR3rst also serves as the floating diffusion FD3.

The amplifier transistor TR3amp includes a gate, a channel formation region, and a source/drain region. The gate is coupled to another source/drain region (floating diffusion FD3) included in the reset transistor TR3rst. One source/drain region included in the amplifier transistor TR3amp shares a region with another source/drain region included in the reset transistor TR3rst, and is coupled to the power line VDD.

The selection transistor TR3sel includes a gate, a channel formation region, and a source/drain region. The gate is coupled to a selection line SEL3. One source/drain region included in the selection transistor TR3sel shares a region with another source/drain region included in the amplifier transistor TR3amp. Another source/drain region included in the selection transistor TR3sel is coupled to a signal line (data output line) VSL3.

The reset lines RST1, RST2, and RST3, the selection lines SEL1, SEL2, and SEL3, and the transfer gate lines TG2 and TG3 are each coupled to a vertical drive circuit included in a drive circuit. The signal lines (data output lines) VSL1, VSL2, and VSL3 are coupled to the column signal processing circuit 112 included in the drive circuit.

### (1-2. Method of Manufacturing Imaging Element)

For example, it is possible to manufacture the imaging element 10 in the present embodiment as follows.

FIGS. 9A to 9K illustrate a method of manufacturing the imaging element 10 in process order. First, as illustrated in FIG. 9A, for example, the p-well 31 is formed in the semiconductor substrate 30, and the n-type photoelectric conversion regions 32B and 32R are formed in this p-well 31, for example. A p+ region is formed near the first surface 30A of the semiconductor substrate 30.

On the second surface 30B of the semiconductor substrate 30, for example, in the same manner as illustrated in FIG. 9A, after forming an n+ region that is to be the floating diffusions FD1 to FD3, the gate insulating layer 33 and a gate wiring layer 47 including the gate of each of the transfer transistor Tr2, the transfer transistor Tr3, the selection transistor SEL, the amplifier transistor AMP, and the reset transistor RST are formed. Accordingly, the transfer transistor Tr2, the transfer transistor Tr3, the selection transistor SEL, the amplifier transistor AMP, and the reset transistor RST are formed. Furthermore, the multilayer wiring layer 40 that include the wiring layers 41 to 43 including the lower first contact 45, the lower second contact 46, and the coupling section 41A and the insulating layer 44 is formed on the second surface 30B of the semiconductor substrate 30.

As a substrate for the semiconductor substrate 30, for example, an SOI (Silicon on Insulator) substrate on which the semiconductor substrate 30, an embedded oxide film (not illustrated), and a holding substrate (not illustrated) are stacked is used. Although not illustrated in FIG. 9A, the embedded oxide film and the holding substrate are bonded to the first surface 30A of the semiconductor substrate 30. After ion implantation, annealing processing is performed.

Next, a support substrate (not illustrated) or another semiconductor substrate or the like is bonded onto the multilayer wiring layer 40 provided on the second surface 30B side of the semiconductor substrate 30, and is vertically inverted. Subsequently, the semiconductor substrate 30 is separated from the embedded oxide film of the SOI substrate and the support substrate, to expose the first surface 30A of the semiconductor substrate 30. It is possible to perform the above process using a technique used in an ordinary CMOS process, such as ion implantation or a CVD (Chemical Vapor Deposition) method.

Next, as illustrated in FIG. 9B, for example, the semiconductor substrate 30 is processed by dry etching from the first surface 30A side to form the opening 34H, for example, in an annular shape. As illustrated in FIG. 9B, the opening 34H has a depth to penetrate from the first surface 30A to the second surface 30B of the semiconductor substrate 30 and reach, for example, the coupling section 41A.

Subsequently, for example, the fixed charge layer 27A and the dielectric layer 27B are formed sequentially on the first surface 30A of the semiconductor substrate 30 and the side surface of the opening 34H. For example, it is possible to form the fixed charge layer 27A by forming a hafnium oxide film or an aluminum oxide film using an atomic layer deposition (ALD) method. It is possible to form the dielectric layer 27B, for example, by forming a silicon oxide film using a plasma CVD method. Next, at a predetermined position on the dielectric layer 27B, for example, the pads 39A, 39B, and 39C in which a barrier metal including a stacked film of titanium and titanium nitride (Ti/TiN film) and a tungsten film are stacked are formed. This makes it possible to use the pads 39A and 39B as a light-shielding film. Then, the interlayer insulating layer 28 is formed on the dielectric layer 27B and on the pads 39A, 39B, and 39C, and the surface of the interlayer insulating layer 28 is planarized using a CMP (Chemical Mechanical Polishing) method.

Subsequently, as illustrated in FIG. 9C, after forming openings 28H1, 28H2 on respective ones of the pads 39A, 39B, and 39C, a conductive material such as Al is embedded in these openings 28H1, 28H2, and 28H3 to form the upper first contact 39D, the upper second contact 39E, and the upper third contact 39F.

Next, as illustrated in FIG. 9D, a conductive film 21x is formed on the interlayer insulating layer 28 using, for example, a sputtering method, and then patterning is performed using a photolithography technique. Specifically, after forming a photoresist PR at a predetermined position on the conductive film 21x, the conductive film 21x is processed using dry etching or wet etching. Then, as illustrated in FIG. 9E, the photoresist PR is removed, thereby forming the readout electrode 21A and the pads 39G and 39H. Then, similarly, on the interlayer insulating layer 28, a lower layer of the insulating layer 22, the upper fourth contact 39I and the upper fifth contact 39J, the transfer electrode 21C and the pad 39K, a middle layer of the insulating layer 22, the upper sixth contact 39L, the storage electrode 21B, and an upper layer of the insulating layer 22 are sequentially formed.

Subsequently, as illustrated in FIG. 9F, the first layer 23A is formed on the insulating layer 22 using, for example, PVD, and then a hard mask 221 is formed on the first layer 23A. Next, as illustrated in FIG. 9G, for example, after forming the opening 22H on the readout electrode 21A using, for example, dry etching, the second layer 23B is formed using, for example, ALD, as illustrated in FIG. 9H. Then, after forming the protective film 29 to fill the opening 22H as illustrated in FIG. 9I, the protective film 29 and the hard mask 221 formed on the first layer 23A are ground using a CMP method to expose the first layer 23A as illustrated in FIG. 9J.

Subsequently, as illustrated in FIG. 9K, the protective layer 24 is formed using, for example, ALD. Then, the photoelectric conversion layer 25 and the upper electrode 26 are formed on the protective layer 24. The photoelectric conversion layer 25 is formed using, for example, a vacuum evaporation method. As with the lower electrode 21, the upper electrode 26 is formed using, for example, a sputtering method. Finally, the protective layer 51 including the wiring 52 and the light shielding film 53, and the on-chip lens 54 are provided on the upper electrode 26. Thus, the imaging element 10 illustrated in FIG. 1 is completed.

It is to be noted that as described above, in a case where another layer including an organic material such as a buffer layer that also serves as an electron blocking film, a buffer layer that also serves as a hole blocking film, a work function adjustment layer or the like is formed between the protective layer 24 and the photoelectric conversion layer 25 and between the photoelectric conversion layer 25 and the upper electrode 26, it is preferable to continuously form each layer in a vacuum process (in an integrated vacuum process). In addition, the method of forming the photoelectric conversion layer 25 is not necessarily limited to the technique using a vacuum evaporation method, but a spin coating technique, a printing technique, or the like may also be used, for example. Furthermore, as a method of forming a transparent electrode (the lower electrode 21 and the upper electrode 26) other than the sputtering method, it is possible to name, depending on the material included in the transparent electrode, a physical vapor deposition (PVD) method such as a vacuum deposition method, a reactive vapor deposition method, an electron beam deposition method, or an ion plating method, a pyrosol method, a method of thermally decomposing an organometallic compound, a spray method, a dip method, and various CVD methods including a MOCVD method, an electroless plating method, and an electroplating method.

### (1-3. Signal Acquisition Operation by Imaging Element)

In the imaging element 10, when light enters the photoelectric conversion section 20 via the on-chip lens 54, the light is transmitted through the photoelectric conversion section 20, and the photoelectric conversion regions 32B and 32R in this order, to be photoelectrically converted for each color light of green (G), blue (B), and red (R) in the transmission process. The following will describe a signal acquisition operation for each color.

### (Acquisition of Green Signal by Photoelectric Conversion Section 20)

Of the incoming light to the imaging element 10, green light is first selectively detected (absorbed) and photoelectrically converted in the photoelectric conversion section 20.

The photoelectric conversion section 20 is coupled to the gate Gamp of the amplifier transistor TR1amp and the floating diffusion FD1 via the through electrode 34. Accordingly, from the exciton generated in the photoelectric conversion section 20, an electron is extracted from the lower electrode 21 side and transferred to the second surface 30S2 side of the semiconductor substrate 30 via the through electrode 34, to be accumulated in the floating diffusion FD1. At the same time, the amount of the electric charge generated in the photoelectric conversion section 20 is modulated into a voltage by the amplifier transistor TR1amp.

In addition, the reset gate Grst of the reset transistor TR1rst is disposed next to the floating diffusion FD1. Accordingly, the carrier accumulated in the floating diffusion FD1 is reset by the reset transistor TR1rst.

Because the photoelectric conversion section 20 is coupled not only to the amplifier transistor TR1amp but also to the floating diffusion FD1 via the through electrode 34, this facilitates the resetting of the carrier accumulated in the floating diffusion FD1 by the reset transistor TR1rst.

In contrast, in a case where the through electrode 34 is not coupled to the floating diffusion FD1, it becomes difficult to reset the carrier accumulated in the floating diffusion FD1, making it necessary to pull out the carrier towards the upper electrode 26 side by applying a large voltage. For this reason, there is a possibility of damage to the photoelectric conversion layer 25. In addition, this configuration has difficulty because the structure that allows resetting in a short time causes an increase in dark noise and results in a trade-off.

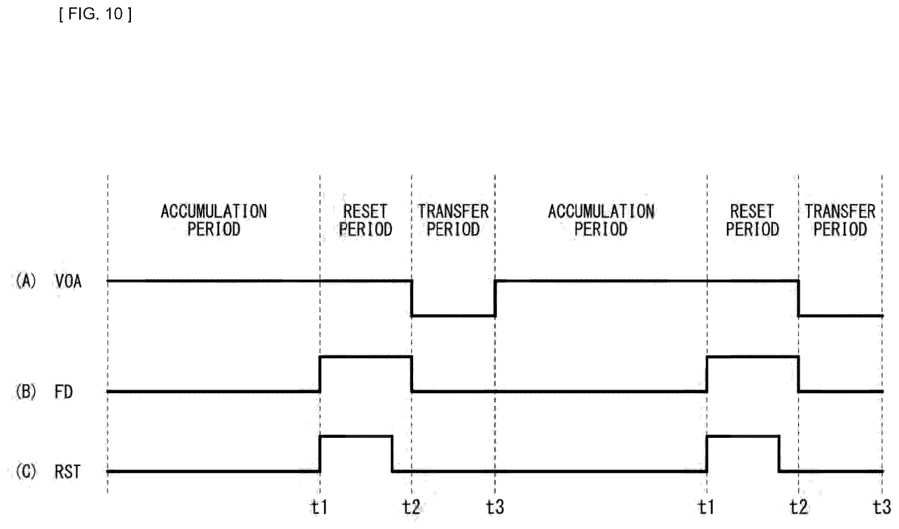

FIG. 10 illustrates an example of an operation of the imaging element 10. (A) illustrates a potential at the storage electrode 21B, (B) illustrates a potential at the floating diffusion FD1 (readout electrode 21A), and (C) illustrates a potential at the gate (Gsel) of the reset transistor TR1rst. In the imaging element 10, a voltage is applied separately to each of the readout electrode 21A and the storage electrode 21B.

In the imaging element 10, during an accumulation period, a potential V1 is applied to the readout electrode 21A from the drive circuit, and a potential V2 is applied to the storage electrode 21B. A potential V6 is applied to the transfer electrode 21C. Here, it is assumed that V1>V6 and V2>V6. This causes the carrier (signal charge; electron) generated by photoelectric conversion to be attracted to the storage electrode 21B and accumulated in a region included in the semiconductor layer 23 and opposed to the storage electrode 21B (accumulation period). Incidentally, a value of the potential at the region included in the semiconductor layer 23 and opposed to the storage electrode 21B becomes more negative with time as the photoelectric conversion proceeds. It is to be noted that the hole is delivered from the upper electrode 26 to the drive circuit.

In the imaging element 10, the reset operation is performed at a later stage in the accumulation period. Specifically, at timing t1, the scanning section changes the voltage of the reset signal RST from a low level to a high level. This causes the reset transistor TR1rst to be turned on in the unit pixel P, and as a result, the voltage of the floating diffusion FD1 is set to the supply voltage, and the voltage of the floating diffusion FD1 is reset (reset period).

After the reset operation is completed, the carrier is read out. Specifically, at timing t2, the drive circuit applies a potential V3 to the readout electrode 21A, applies a potential V4 to the storage electrode 21B, and applies a potential V5 to the transfer electrode 21C. Here, it is assumed that the potentials V3, V4, and V5 are V3>V5>V4. This cause the carrier accumulated above the storage electrode 21B to move from on the storage electrode 21B to on the transfer electrode 21C, and to on the readout electrode 21A in this order, to be read out to the floating diffusion FD1. In other words, the carrier accumulated in the semiconductor layer 23 is read out to the control section (transfer period).

After the readout operation is completed, the drive circuit again applies the potential V1 to the readout electrode 21A, and applies the potential V2 to the storage electrode 21B. This causes the carrier generated by photoelectric conversion to be attracted to the storage electrode 21B and accumulated in a region included in the photoelectric conversion layer 25 and opposed to the storage electrode 21B (accumulation period).

### (Acquisition of Blue Signal and Red Signal by the Photoelectric Conversion Regions 32B and 32R)

Subsequently, from the light transmitted through the photoelectric conversion section 20, blue light and red light are sequentially absorbed and photoelectrically converted in the photoelectric conversion region 32B and 32R, respectively. In the photoelectric conversion region 32B, an electron corresponding to the incoming blue light is accumulated in an n-region in the photoelectric conversion region 32B, and the accumulated electron is transferred to the floating diffusion FD2 by the transfer transistor Tr2. Similarly, in the photoelectric conversion region 32R, an electron corresponding to the incoming red light is accumulated in an n-region in the photoelectric conversion region 32R, and the accumulated electron is transferred to the floating diffusion FD3 by the transfer transistor Tr3.

### (1-4. Workings and Effects)

In the imaging element 10 of the present embodiment, the photoelectric conversion section 20 includes the semiconductor layer 23 between the lower electrode 21 including the readout electrode 21A and the storage electrode 21B and the photoelectric conversion layer 25, and the semiconductor layer 23 includes the first layer 23A and the second layer 23B having a difference from each other, above the readout electrode 21A and the storage electrode 21B, respectively. The difference between the first layer 23A and the second layer 23B is in at least one of material composition, crystallinity, impurity concentration contained, or constituent element. This will be described below.

In recent years, as an imaging element included in a CCD image sensor, a CMOS image sensor, or the like, development of a stacked type imaging element in which a plurality of photoelectric conversion sections is vertically stacked has been promoted. For example, the stacked type imaging element has a configuration in which two photoelectric conversion regions each including a photodiode (PD) are stacked to be included in a silicon (Si) substrate, and a photoelectric conversion layer including an organic material is provided above the Si substrate.

In a stacked type imaging element, it is necessary to have a configuration to accumulate and transfer the signal charge generated in each photoelectric conversion section. For example, the photoelectric conversion section is enabled to accumulate the signal charge generated in the photoelectric conversion layer by having a configuration in which two electrodes, the first electrode and a charge storage electrode, are provided on a photoelectric conversion region side of a pair of electrodes arranged opposed to each other with the photoelectric conversion layer interposed therebetween. In such an imaging device, the signal charge is once accumulated above the charge storage electrode and then transferred to the floating diffusion FD in the Si substrate. This allows the electric charge accumulation section to be completely depleted when exposure starts, thereby erasing the carrier. As a result, it is possible to suppress such a phenomenon as an increase in kTC noise, deterioration of random noise, or degradation of image quality.

However, a solid-state imaging device of a stacked type also has a difficulty. It takes time to transfer the accumulated charge from the charge storage electrode to the readout electrode due to the configuration in which the charge accumulated in the semiconductor layer on the charge storage electrode is once caused to move laterally along the upper surface of the charge storage electrode and then flow into the readout electrode located below the charge storage electrode. For this reason, it is difficult to increase a reading speed for reading a pixel signal from each pixel. In response to these difficulties, a configuration has been proposed in which the transfer of an electric charge within the semiconductor layer for electric charge accumulation is arranged vertically, as described earlier. In this proposal, the charge storage electrode is arranged to allow for a vertical transfer path of the electric charge in the semiconductor layer stacked under the photoelectric conversion layer.

Meanwhile, in the semiconductor layer that plays a role in electric charge accumulation, it is important to use a semiconductor film having few traps. It is known that an electron caught in a trap during electric charge accumulation is released with a delay during electric charge transfer, and becomes a noise source as a transfer delay charge.

In contrast, in the present embodiment, as the semiconductor layer 23 provided between the lower electrode 21 and the photoelectric conversion layer 25, the first layer 23A and the second layer 23B having a difference in at least one of material composition, crystallinity, impurity concentration contained, or constituent element are provided. This makes it possible to achieve both low trapping above the storage electrode 21B and embeddability in the opening 22H, thereby improving a transport characteristic of the carrier to the readout electrode 21A.

As described above, for the imaging element 10 of the present embodiment, it is possible to improve image quality.

In addition, in the imaging element 10 of the present embodiment, the readout electrode 21A and the storage electrode 21B are provided on different layers. Specifically, the storage electrode 21B is provided in an upper layer than the readout electrode 21A, the first layer 23A described above is provided above the storage electrode 21B, and the second layer 23B described above is provided above the readout electrode 21A. This makes it possible to perform a control to cause the electric charge to be accumulated above the storage electrode 21B even when the electron to be the signal charge reaches the second layer 23B on the readout electrode 21A, thereby making it possible to reduce a loss of electric charge. Thus, it is possible to increase an area of the storage electrode 21B.

Next, some modification examples (Modification examples 1-6) of the present disclosure will be described. In the following, components similar to those in the above embodiment will be denoted by the same reference numerals and the description thereof will be omitted as appropriate.

### 2. Modification Examples

### 2-1. Modification Example 1

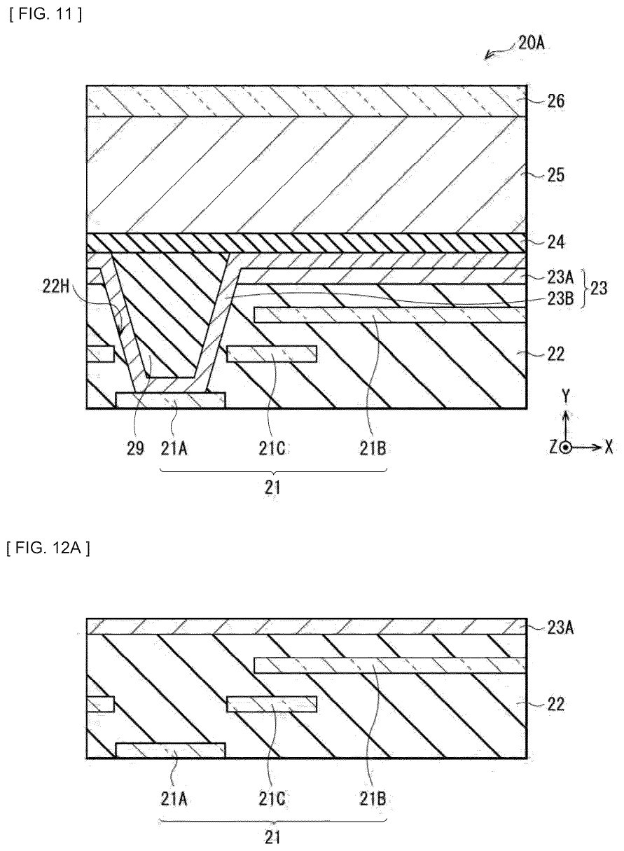

FIG. 11 schematically illustrates a cross-sectional configuration of a main section (photoelectric conversion section 20A) of an imaging element of Modification example 1 of the present disclosure. The photoelectric conversion section 20A of the present modification example differs from the above embodiment in that the second layer 23B is extended over the first layer 23A.

For example, it is possible to manufacture the photoelectric conversion section 20A of the present modification example as follows.

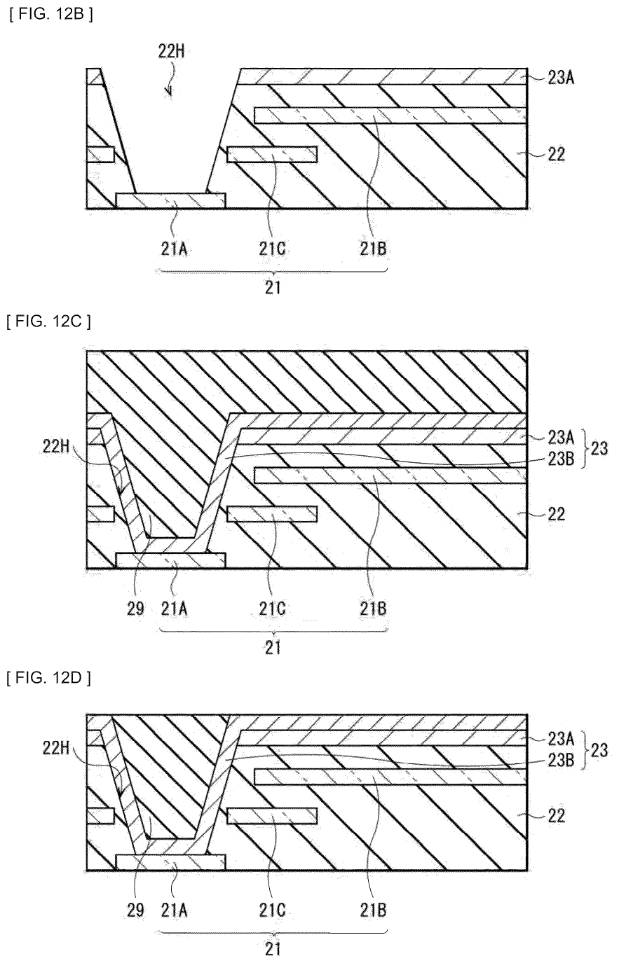

As in the above embodiment, as illustrated in FIG. 12A, the first layer 23A is formed on the insulating layer 22 using, for example, PVD. Next, as illustrated in FIG. 12B, after forming the opening 22H on the readout electrode 21A using, for example, dry etching, the second layer 23B is formed using, for example, ALD. Then, as illustrated in FIG. 12C, after forming the protective film 29 to fill the opening 22H, the protective film 29 formed on the first layer 23A is ground using a CMP method to expose the second layer 23B, as illustrated in FIG. 12D. Then, the protective layer 24, the photoelectric conversion layer 25, and the upper electrode 26 are formed as in the above embodiment. Thus, the imaging element 10 illustrated in FIG. 11 is completed.

In this manner, in the present modification example, the second layer 23B is extended over the first layer 23A to cause the first layer 23A and the second layer 23B to be stacked above the storage electrode 21B. The carrier (electron) generated in the photoelectric conversion layer 25 is accumulated in the semiconductor layer 23 above the storage electrode 21B, but most of the carrier is accumulated in the semiconductor layer 23 near an interface with the insulating layer 22, namely, the first layer 23A. For this reason, there is a small trapping effect on the second layer 23B, and therefore it is possible to obtain an effect similar to that of the above embodiment.

### 2-2. Modification Example 2

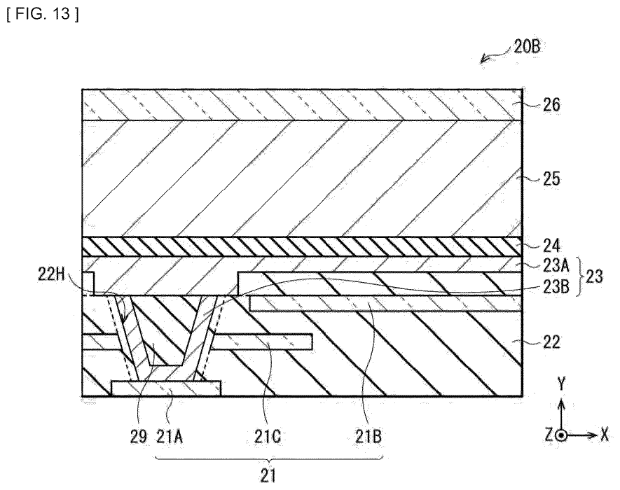

FIG. 13 schematically illustrates a cross-sectional configuration of a main section (photoelectric conversion section 20B) of an imaging element of Modification example 2 of the present disclosure. The photoelectric conversion section 20B of the present modification example differs from the above embodiment in that the first layer 23A is formed across an entire surface and extended to be over the readout electrode 21A.

For example, it is possible to manufacture the photoelectric conversion section 20A of the present modification example as follows.

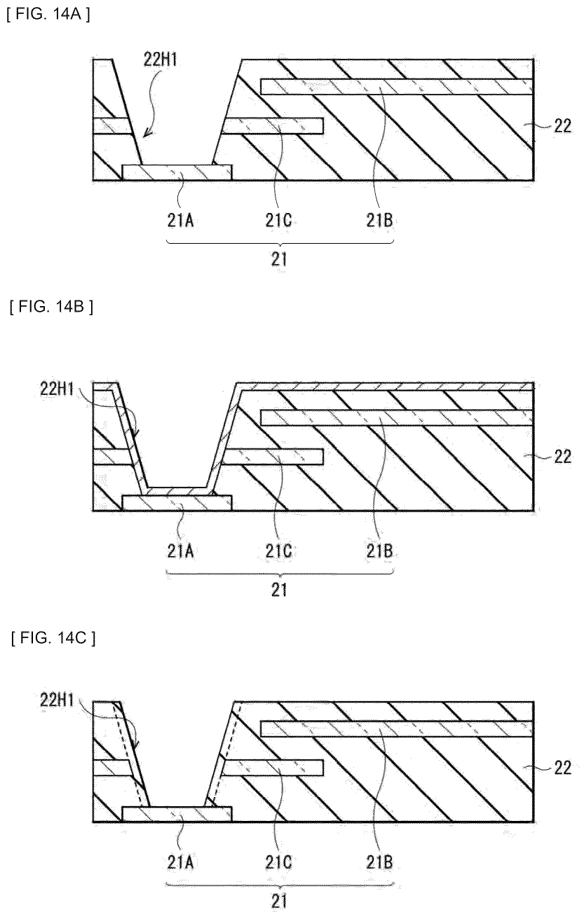

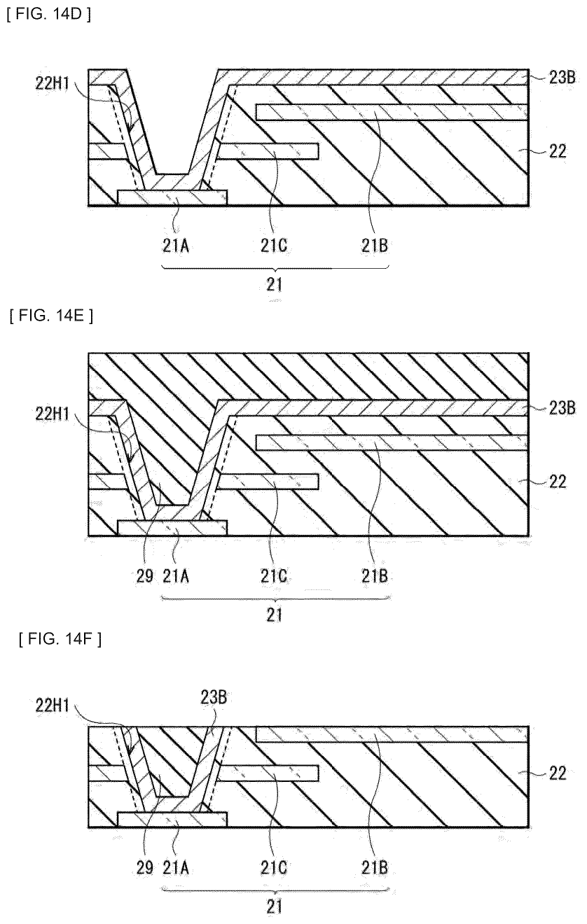

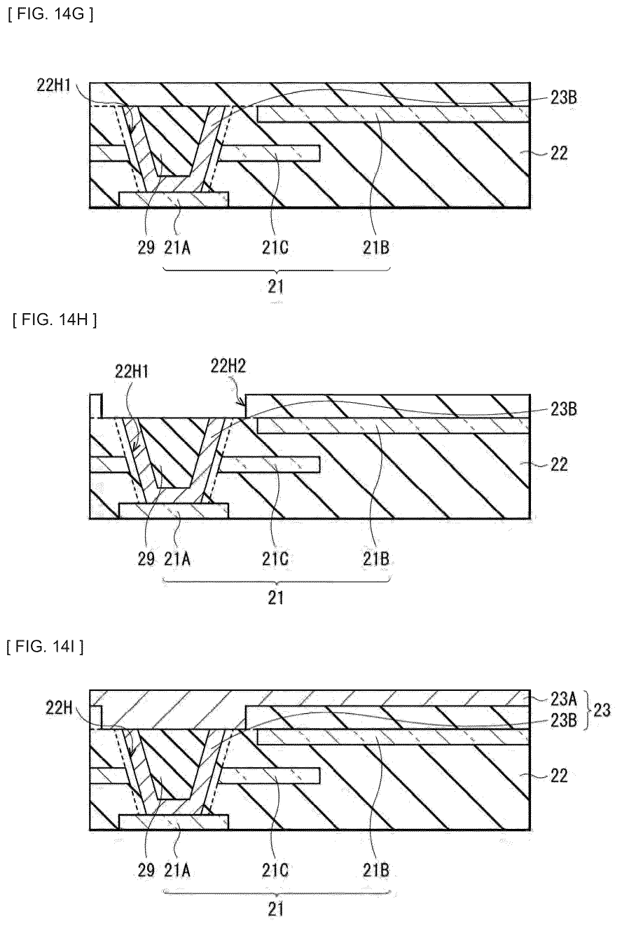

As in the above embodiment, as illustrated in FIG. 14A, the insulating layer 22 is formed to cover the storage electrode 21B. Next, as illustrated in FIG. 14B, for example, after forming an opening 22H1 on the readout electrode 21A using dry etching, the insulating layer 22 is formed again to cover a side surface and a bottom surface of the opening 22H1. Subsequently, as illustrated in FIG. 14C, after removing the insulating layer 22 on the readout electrode 21A by etching back, the second layer 23B is formed using, for example, ALD as illustrated in FIG. 14D.

Next, as illustrated in FIG. 14E, after forming the protective film 29 to fill the opening 22H1, the protective film 29 formed on the second layer 23B is ground using a CMP method to expose the second layer 23B, as illustrated in FIG. 14F. Subsequently, as illustrated in FIG. 14G, after forming the insulating layer 22 again, an opening 22H2 is formed in the readout electrode 21A, as illustrated in FIG. 14H. Next, as illustrated in FIG. 14I, the first layer 23A is formed on the insulating layer 22 to fill the opening 22H2 using, for example, PVD. Then, the protective layer 24, the photoelectric conversion layer 25, and the upper electrode 26 are formed as in the above embodiment. Thus, the imaging element 10 illustrated in FIG. 13 is completed.

In this manner, in the present modification example, a distance between the transfer electrode 21C and the semiconductor layer 23B is precisely controlled compared to the above embodiment, etc. Accordingly, in addition to the effects of the above embodiment, it is possible to expand a margin for voltage setting.

### 2-3. Modification Example 3

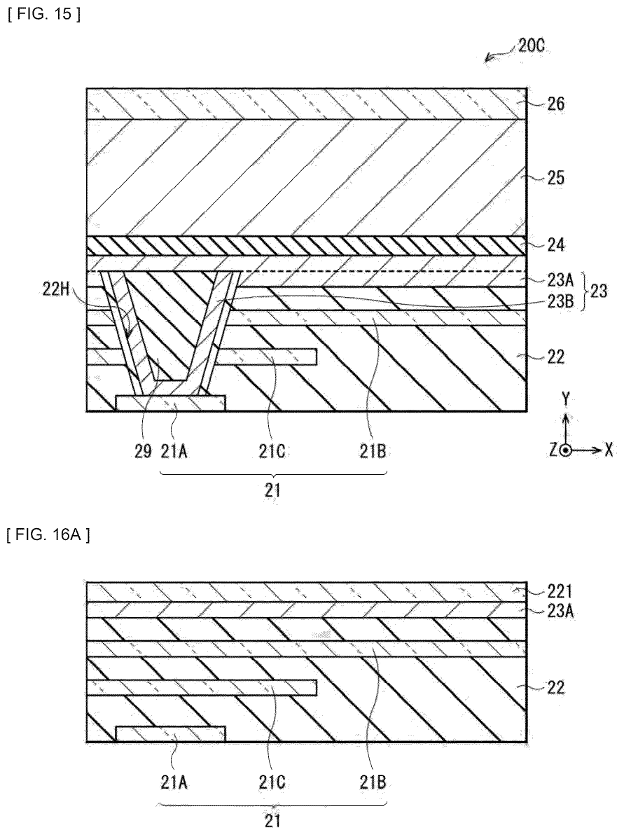

FIG. 15 schematically illustrates a cross-sectional configuration of a main section (photoelectric conversion section 20C) of an imaging element in Modification example 3 of the present disclosure. As in the above Modification example 2, in the photoelectric conversion section 20C of the present modification example, the first layer 23A is formed across an entire surface and extended to be over the readout electrode 21A.

For example, it is possible to manufacture the photoelectric conversion section 20A in the present modification example as follows.

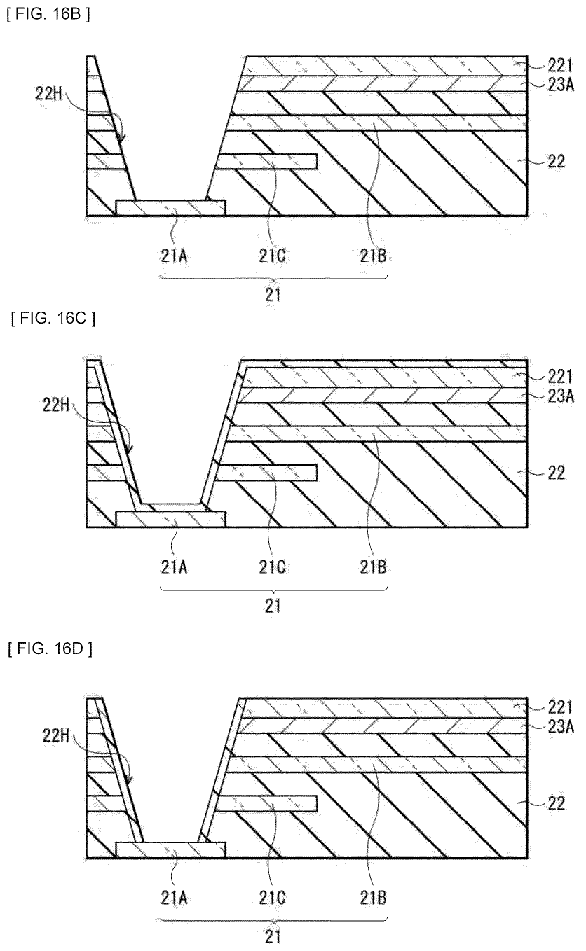

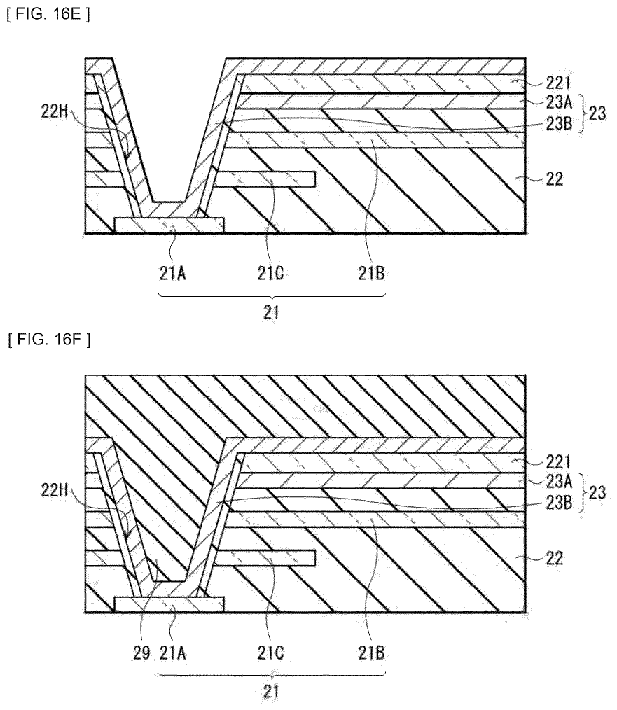

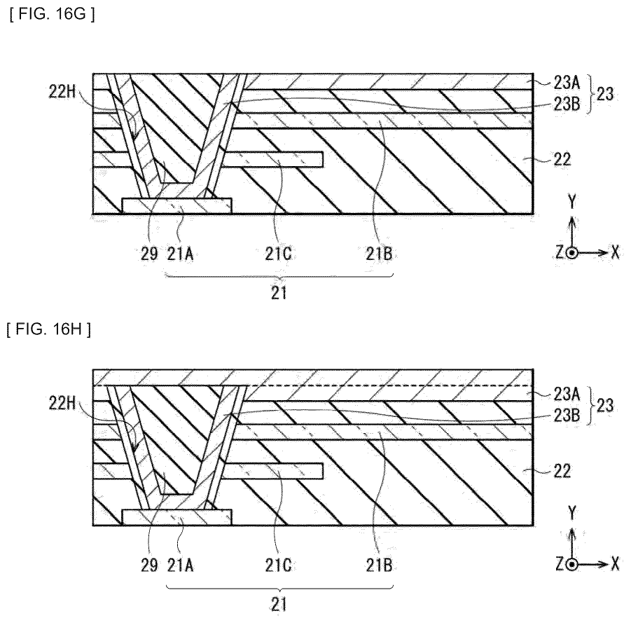

As in the above embodiment, as illustrated in FIG. 16A, after forming the first layer 23A on the insulating layer 22 using, for example, PVD, a hard mask 221 is formed on the first layer 23A. Next, as illustrated in FIG. 16B, after forming the opening 22H on the readout electrode 21A using, for example, dry etching, the insulating layer 22 is formed again to cover a side surface and a bottom surface of the opening 22H1 as illustrated in FIG. 16C. Subsequently, as illustrated in FIG. 16D, after removing the insulating layer 22 on the readout electrode 21A by etching back, the second layer 23B is formed using, for example, ALD as illustrated in FIG. 16E.

Next, as illustrated in FIG. 16F, after forming the protective film 29 to fill the opening 22H, the protective film 29 formed on the second layer 23B is ground using the CMP method to expose the first layer 23A as illustrated in FIG. 16G. Subsequently, as illustrated in FIG. 16H, the first layer 23A is formed again. Then, the protective layer 24, the photoelectric conversion layer 25, and the upper electrode 26 are formed as in the above embodiment. Thus, the imaging element 10 illustrated in FIG. 15 is completed.

In this manner, in the method of manufacturing the present modification example, it is not necessary to include a process for forming an opening on the readout electrode 21A, thus allowing a process more compatible with miniaturization.

Furthermore, the present technique is applicable to an imaging element having the following configuration.

### 2-4. Modification Example 4

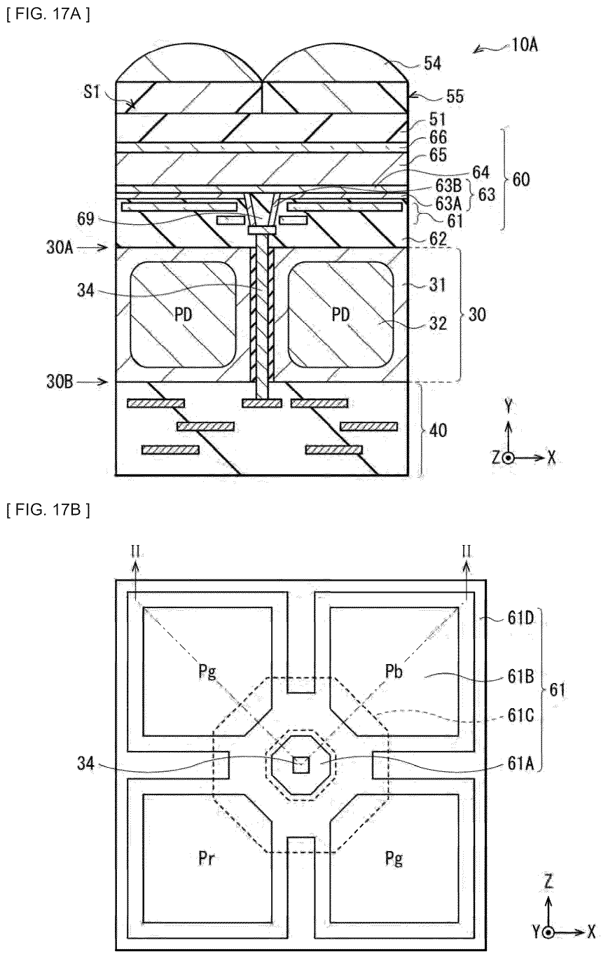

FIG. 17A schematically illustrates a cross-sectional configuration of an imaging element 10A of Modification example 4 of the present disclosure. FIG. 17B schematically illustrates an example of a planar configuration of the imaging element 10A illustrated in FIG. 17A, and FIG. 17A illustrates a cross section at a line II-II illustrated in FIG. 17B. For example, the imaging element 10A is a stacked type imaging element in which the photoelectric conversion region 32 and the photoelectric conversion section 60 are stacked.

In the imaging element 10A of the present modification example, above the photoelectric conversion section 60 (on the light incoming side S1), a color filter 55 that selectively transmits red light (R), green light (G), and blue light (B) is provided for each unit pixel P. Specifically, in a pixel unit 1a, which includes four pixels arranged in two rows by two columns, two color filters that selectively transmit green light (G) are arranged on a diagonal line, and color filters that selectively transmit red light (R) and blue light (B) are arranged one each on a diagonal line orthogonal to the previous one. For example, in the unit pixel (Pr, Pg, and Pb) each including a color filter, light of each corresponding color is detected in the photoelectric conversion section 60. In other words, in the pixel section 1A, the pixels (Pr, Pg, and Pb) that detect red light (R), green light (G), and blue light (B), respectively, are arranged in a Bayer layout.

For example, the photoelectric conversion section 60 includes a lower electrode 61 including a readout electrode 61A, a storage electrode 62B, a transfer electrode 61C, and a shield electrode 61D, an insulating layer 62, a semiconductor layer 63 including a first layer 63A and a second layer 63B, a protective layer 64, a photoelectric conversion layer 65, and an upper electrode 66. Each of the lower electrode 61, the insulating layer 62, the semiconductor layer 63, the photoelectric conversion layer 65, and the upper electrode 66 has a similar configuration to that of the photoelectric conversion section 20 in the above embodiment. The photoelectric conversion region 32 detects light having a wavelength range different from that of the photoelectric conversion section 60.

In the imaging element 10A, from the light transmitted through the color filter 55, light of a visible light region (red light (R), green light (G), and blue light (B)) is absorbed by the photoelectric conversion section 60 in the unit pixel (Pr, Pg, and Pb) including each color filter, and other light, for example, light of an infrared light region (for example, light (infrared light (IR)) of 700 nm or more and 1000 nm or less, is transmitted through the photoelectric conversion section 60. The infrared light (IR) transmitted through the photoelectric conversion section 60 is detected in the photoelectric conversion region 32 in each unit pixel Pr, Pg, or Pb, and a signal charge corresponding to the infrared light (IR) is generated in each unit pixel Pr, Pg, or Pb. In other words, it becomes possible for the imaging device 1 including the imaging element 10A to simultaneously generate both a visible light image and an infrared light image.

It is to be noted that the above embodiment illustrates, but is not limited to, an example in which the readout electrode 21A is provided for each unit pixel P. For example, as illustrated in FIG. 17B, the readout electrode 21A may be formed one each in an approximate center of the pixel unit 1a, for example, assuming that the pixel unit 1a including four unit pixels P arranged in two rows by two columns is a repeating unit.

### 2-5. Modification Example 5

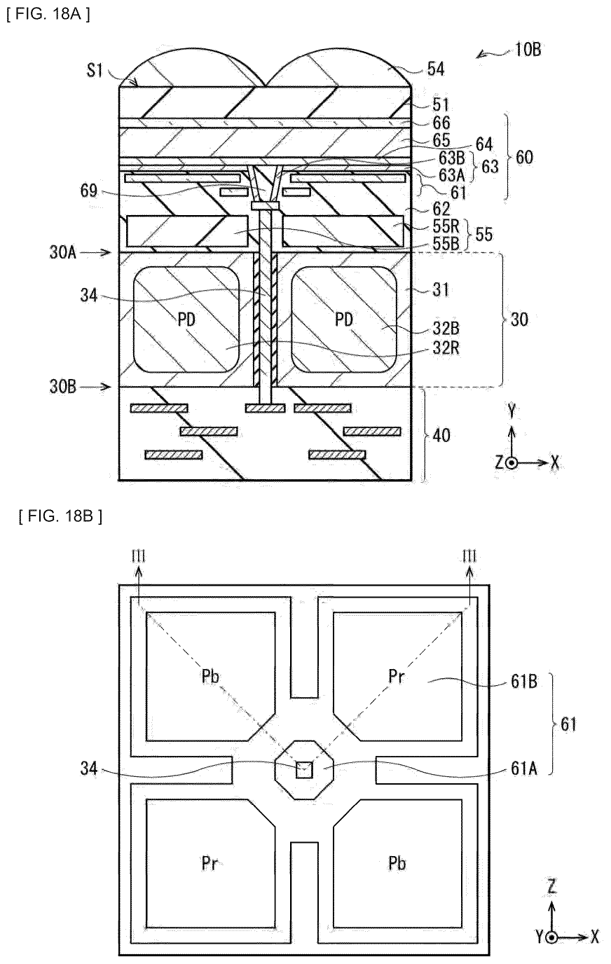

FIG. 18A schematically illustrates a cross-sectional configuration of an imaging element 10B of Modification example 5 of the present disclosure. FIG. 18B schematically illustrates an example of a planar configuration of the imaging element 10B illustrated in FIG. 18A, and FIG. 18A illustrates a cross section at a line III-III illustrated in FIG. 18B. In the above Modification example 7, the color filter 55 that selectively transmits red light (R), green light (G), and blue light (B) is provided above the photoelectric conversion section 60 (light incoming side S1). However, as illustrated in FIG. 18A, for example, the color filter 55 may be provided between the photoelectric conversion region 32 and the photoelectric conversion section 60.

For example, in the imaging element 10B, the color filter 55 has a configuration in which, within the pixel unit 1a, a color filter (color filter 55R) that selectively transmits at least red light (R) and a color filter (color filter 55B) that selectively transmits at least blue light (B) are arranged diagonally to each other. The photoelectric conversion section 60 (photoelectric conversion layer 65) is configured to selectively absorb, for example, a wavelength corresponding to green light as in the above embodiment. This makes it possible to acquire a signal corresponding to RGB in the photoelectric conversion regions (photoelectric conversion regions 32R and 32G) each provided below a corresponding one of the photoelectric conversion section 60 and the color filters 55R and 55B. The imaging element 10B of the present modification example makes it possible to increase an area of each photoelectric conversion section in RGB compared to an imaging element having a general Bayer layout, thus making it possible to improve an S/N ratio.

### 2-6. Modification Example 6

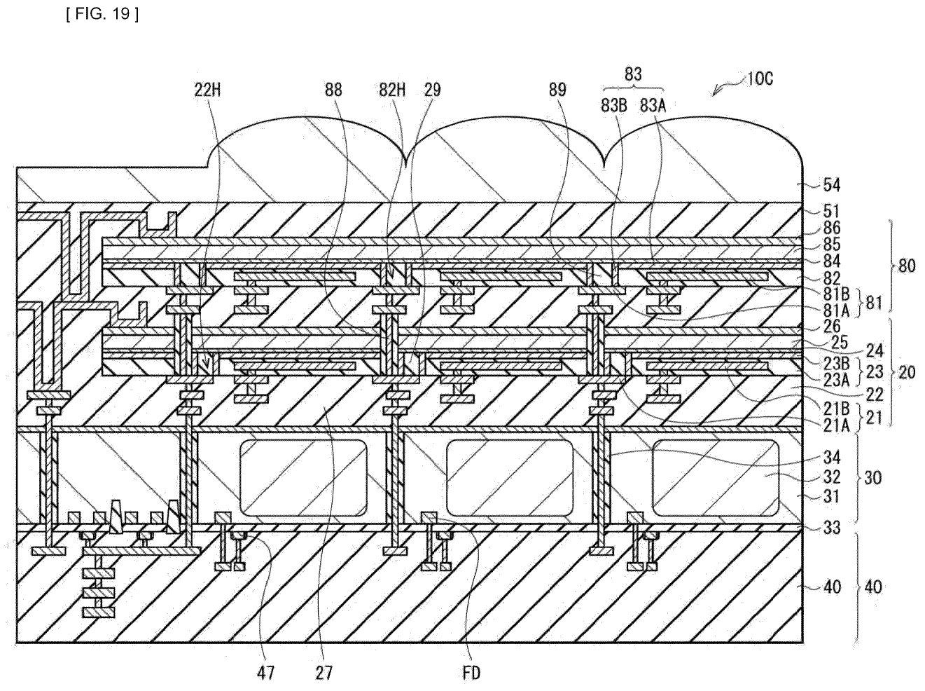

FIG. 19 schematically illustrates a cross-sectional configuration of an imaging element 10C according to Modification example 6 of the present disclosure. In the imaging element 10C of the present modification example, two photoelectric conversion sections 20 and 80 and one photoelectric conversion region 32 are vertically stacked.

The photoelectric conversion sections 20 and 80 and the photoelectric conversion region 32 selectively detect light having a wavelength range different from each other and perform photoelectric conversion. For example, the photoelectric conversion section 20 acquires a green (G) signal. For example, the photoelectric conversion section 80 acquires a blue (B) signal. For example, the photoelectric conversion region 32 acquires a red (R) signal. This allows the imaging element 10C to acquire a plurality of types of color signals in a single pixel without using a color filter.

For example, the photoelectric conversion section 80 is stacked above the photoelectric conversion section 20, and, as with the photoelectric conversion section 20, has a configuration in which a lower electrode 81, and for example, a semiconductor layer 83 including a first layer 83A and a second layer 83B, a protective layer 84, a photoelectric conversion layer 85, and an upper electrode 86 are stacked in this order from the first surface 30A side of the semiconductor substrate 30. As in the photoelectric conversion section 20, the lower electrode 81 includes a readout electrode 81A and a storage electrode 81B, which are electrically separated by an insulating layer 82. The insulating layer 82 has an opening 82H on the readout electrode 81A. Between the photoelectric conversion section 80 and the photoelectric conversion section 20, an interlayer insulating layer 87 is provided.

A through electrode 88 is coupled to the readout electrode 81A and penetrates the interlayer insulating layer 87 and the photoelectric conversion section 20 to be electrically coupled to the readout electrode 21A in the photoelectric conversion section 20. Furthermore, the readout electrode 81A is electrically coupled via the through electrodes 34 and 88 to the floating diffusion FD provided on the semiconductor substrate 30, thereby making it possible to temporarily accumulate the carrier generated in the photoelectric conversion layer 85. Furthermore, the readout electrode 81A is electrically coupled via the through electrodes 34 and 88 to the amplifier transistor AMP or the like provided on the semiconductor substrate 30.

### 3. Application Examples

### Application Example 2

FIG. 20 illustrates an example of an overall configuration of an imaging device (imaging device 1) that includes the imaging element (for example, imaging element 10) illustrated in FIG. 1, etc.

For example, the imaging device 1 is a CMOS image sensor and captures the incoming light (image light) from a subject via an optical lens system (not illustrated), converts an amount of the incoming light formed into an image on an imaging surface into an electrical signal on a pixel-by-pixel basis, and outputs the electrical signal as a pixel signal. The imaging device 1 includes a pixel section 100A as an imaging area on the semiconductor substrate 30, and also includes, for example, a vertical drive circuit 111, a column signal processing circuit 112, a horizontal drive circuit 113, an output circuit 114, a control circuit 115, and an input/output terminal 116 in a peripheral region of this pixel section 100A.

For example, the pixel section 100A includes a plurality of unit pixels P arranged two-dimensionally in a matrix. In each of these unit pixels P, for example, a pixel drive line Lread (specifically, a row selection line and a reset control line) is provided per pixel row, and a vertical signal line Lsig is provided per pixel column. The pixel drive line Lread transmits a drive signal for reading a signal from a pixel. The pixel drive line Lread has one end coupled to an output terminal corresponding to each row of the vertical drive circuit 111.

The vertical drive circuit 111 is a pixel drive section that includes a shift register, an address decoder, or the like, and drives, for example, each unit pixel P of the pixel section 100A on a row-by-row basis. The signal outputted from each unit pixel P in a pixel row selected and scanned by the vertical drive circuit 111 is supplied to the column signal processing circuit 112 through each vertical signal line Lsig. The column signal processing circuit 112 includes an amplifier, a horizontal selection switch, or the like that is provided for each vertical signal line Lsig.

The horizontal drive circuit 113 includes a shift register, an address decoder, or the like, and sequentially drives each horizontal selection switch in the column signal processing circuit 112 while scanning. With this selective scanning by the horizontal drive circuit 113, each pixel signal to be transmitted through each vertical signal line Lsig is caused to be sequentially outputted to the horizontal signal line 121 and transmitted to an outside of the semiconductor substrate 30 through the horizontal signal line 121.

The output circuit 114 performs signal processing on the signal sequentially supplied from each column signal processing circuit 112 via the horizontal signal line 121, and outputs the signal. For example, the output circuit 114 performs only buffering in some cases, or performs black level adjustment, column variation compensation, and various digital signal processing or the like in other cases.

A circuit portion including the vertical drive circuit 111, the column signal processing circuit 112, the horizontal drive circuit 113, the horizontal signal line 121, and the output circuit 114 may be formed directly on the semiconductor substrate 30 or may be provided in an external control IC. In addition, these circuit portions may also be formed on another substrate coupled by a cable or the like.

The control circuit 115 receives a clock, data commanding an operation mode, or the like supplied from outside the semiconductor substrate 30, and also outputs data such as internal information of the imaging device 1. The control circuit 115 further includes a timing generator that generates various timing signals, and performs drive control of a peripheral circuit such as the vertical drive circuit 111, the column signal processing circuit 112, and the horizontal drive circuit 113 or the like on the basis of the various timing signals generated by the timing generator.

The input/output terminal 116 exchanges signals with the outside.

### Application Example 2

In addition, for example, the imaging device 1 as described above is applicable to various electronic apparatuses, including an imaging system such as a digital still camera or a digital video camera, a cell phone equipped with an imaging function, or another apparatus equipped with an imaging function.

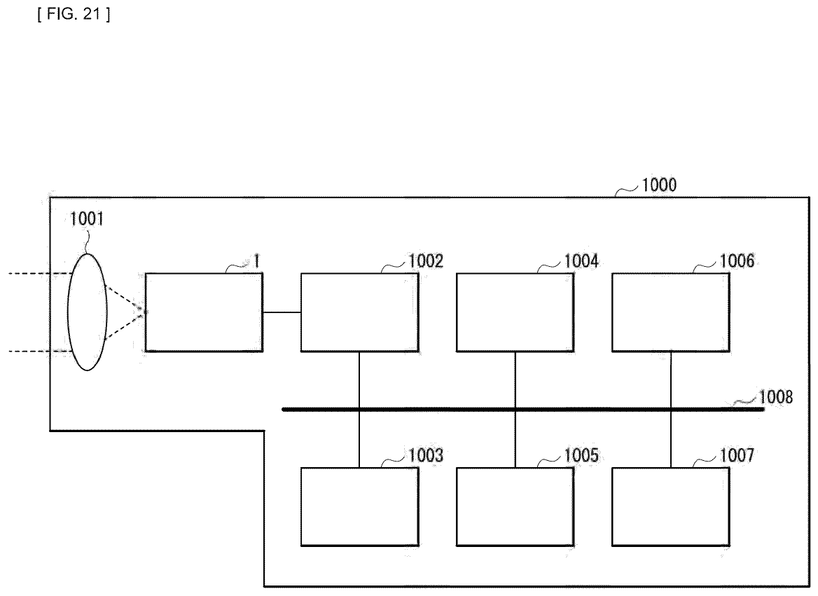

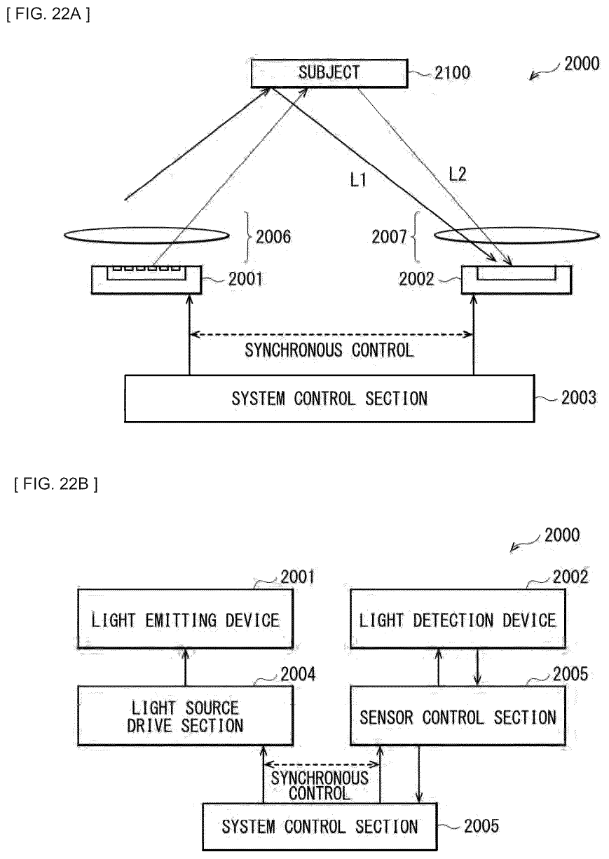

FIG. 21 is a block diagram illustrating an example of a configuration of an electronic apparatus 1000.

As illustrated in FIG. 21, the electronic apparatus 1000 includes an optical system 1001, the imaging device 1, and a DSP (Digital Signal Processor) 1002, and has a configuration in which the DSP 1002, a memory 1003, a display device 1004, a recording device 1005, an operating system 1006, and a power supply system 1007 are coupled via a bus 1008 to enable capture of a still image and a moving image.

The optical system 1001 includes one or a plurality of lenses and captures the incoming light (image light) from a subject to form an image on an imaging surface of the imaging device 1.

For the imaging device 1, the imaging device 1 described above is applied. The imaging device 1 converts an amount of the incoming light formed into an image on the imaging surface by the optical system 1001 into an electrical signal on a pixel-by-pixel basis, and supplies the electrical signal to the DSP 1002 as a pixel signal.

The DSP 1002 obtains an image by performing various signal processing on the signal from the imaging device 1, and causes the memory 1003 to temporarily hold the data of the image. The data of the image held by the memory 1003 is recorded in the recording device 1005 or supplied to the display device 1004 to display the image. In addition, the operating system 1006 accepts various operations performed by a user and supplies an operation signal to each block in the electronic apparatus 1000, and the power supply system 1007 supplies an electric power necessary to drive each block in the electronic apparatus 1000.

### Application Example 3

FIG. 22A schematically illustrates an example of an overall configuration of a light detection system 2000 including the imaging device 1. FIG. 22B illustrates an example of a circuit configuration of the light detection system 2000. The light detection system 2000 includes a light emitting device 2001 as a light source section that emits infrared light L2 and a light detection device 2002 as a light receiving section including a photoelectric conversion element. As the light detection device 2002, it is possible to use the imaging device 1 described above. The light detection system 2000 may further include a system control section 2003, a light source drive section 2004, a sensor control section 2005, a light source side optical system 2006, and a camera side optical system 2007.

For the light detection device 2002, it is possible to detect light L1 and light L2. The light L1 is ambient light from outside reflected by a subject (measurement target) 2100 (FIG. 22A). The light L2 is the light emitted by the light emitting device 2001 and then reflected by the subject 2100. For example, the light L1 is visible light, and the light L2 is, for example, infrared light. The light L1 is detectable in the photoelectric conversion section in the light detection device 2002, and the light L2 is detectable in the photoelectric conversion region in the light detection device 2002. It is possible to obtain image information regarding the subject 2100 from the light L1, and to obtain distance information between the subject 2100 and the light detection system 2000 from the light L2. For example, the light detection system 2000 is mountable on an electronic apparatus such as a smartphone or on a mobile body such as a car. In the light emitting device 2001, it is possible to include, for example, a semiconductor laser, a surface emitting semiconductor laser, or a vertical-cavity surface-emitting laser (VCSEL). For example, as a method of detecting, by the light detection device 2002, the light L2 emitted from the light emitting device 2001, it is possible to adopt, but not limited to, an iTOF method. For example, the iTOF allows the photoelectric conversion section to measure a distance to the subject 2100 by using optical time-of-flight (TOF). For example, as the method of detecting, by the light detection device 2002, the light L2 emitted from the light emitting device 2001, it is also possible to adopt a structured light method or a stereo vision method. For example, the structured light method makes it possible to measure the distance between the light detection system 2000 and the subject 2100 by projecting light having a predetermined pattern onto the subject 2100 and analyzing a distortion level of the pattern. In addition, for example, the stereo vision method makes it possible to measure the distance between the light detection system 2000 and the subject 2100 by capturing two or more images of the subject 2100 using two or more cameras from two or more different viewpoints. It is to be noted that it is possible to synchronously control the light emitting device 2001 and the light detection device 2002 by the system control section 2003.

### 4. Practical Applications

### (Practical Application to Endoscopic Surgical System)

A technique of the present disclosure (the present technique) is applicable to various products. For example, the technique of the present disclosure may be applied to an endoscopic surgical system.

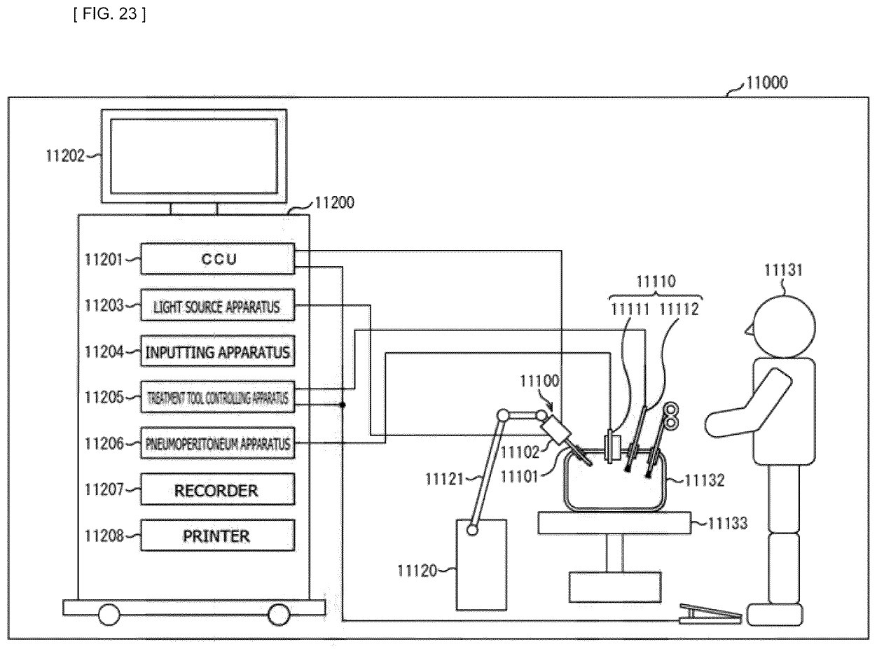

FIG. 23 is a view depicting an example of a schematic configuration of an endoscopic surgery system to which the technology according to an embodiment of the present disclosure (present technology) can be applied.

In FIG. 23, a state is illustrated in which a surgeon (medical doctor) 11131 is using an endoscopic surgery system 11000 to perform surgery for a patient 11132 on a patient bed 11133. As depicted, the endoscopic surgery system 11000 includes an endoscope 11100, other surgical tools 11110 such as a pneumoperitoneum tube 11111 and an energy device 11112, a supporting arm apparatus 11120 which supports the endoscope 11100 thereon, and a cart 11200 on which various apparatus for endoscopic surgery are mounted.

The endoscope 11100 includes a lens barrel 11101 having a region of a predetermined length from a distal end thereof to be inserted into a body cavity of the patient 11132, and a camera head 11102 connected to a proximal end of the lens barrel 11101. In the example depicted, the endoscope 11100 is depicted which includes as a rigid endoscope having the lens barrel 11101 of the hard type. However, the endoscope 11100 may otherwise be included as a flexible endoscope having the lens barrel 11101 of the flexible type.

The lens barrel 11101 has, at a distal end thereof, an opening in which an objective lens is fitted. A light source apparatus 11203 is connected to the endoscope 11100 such that light generated by the light source apparatus 11203 is introduced to a distal end of the lens barrel 11101 by a light guide extending in the inside of the lens barrel 11101 and is irradiated toward an observation target in a body cavity of the patient 11132 through the objective lens. It is to be noted that the endoscope 11100 may be a forward-viewing endoscope or may be an oblique-viewing endoscope or a side-viewing endoscope.

An optical system and an image pickup element are provided in the inside of the camera head 11102 such that reflected light (observation light) from the observation target is condensed on the image pickup element by the optical system. The observation light is photo-electrically converted by the image pickup element to generate an electric signal corresponding to the observation light, namely, an image signal corresponding to an observation image. The image signal is transmitted as RAW data to a CCU 11201.

The CCU 11201 includes a central processing unit (CPU), a graphics processing unit (GPU) or the like and integrally controls operation of the endoscope 11100 and a display apparatus 11202. Further, the CCU 11201 receives an image signal from the camera head 11102 and performs, for the image signal, various image processes for displaying an image based on the image signal such as, for example, a development process (demosaic process).

The display apparatus 11202 displays thereon an image based on an image signal, for which the image processes have been performed by the CCU 11201, under the control of the CCU 11201.

The light source apparatus 11203 includes a light source such as, for example, a light emitting diode (LED) and supplies irradiation light upon imaging of a surgical region to the endoscope 11100.

An inputting apparatus 11204 is an input interface for the endoscopic surgery system 11000. A user can perform inputting of various kinds of information or instruction inputting to the endoscopic surgery system 11000 through the inputting apparatus 11204. For example, the user would input an instruction or a like to change an image pickup condition (type of irradiation light, magnification, focal distance or the like) by the endoscope 11100.

A treatment tool controlling apparatus 11205 controls driving of the energy device 11112 for cautery or incision of a tissue, sealing of a blood vessel or the like. A pneumoperitoneum apparatus 11206 feeds gas into a body cavity of the patient 11132 through the pneumoperitoneum tube 11111 to inflate the body cavity in order to secure the field of view of the endoscope 11100 and secure the working space for the surgeon. A recorder 11207 is an apparatus capable of recording various kinds of information relating to surgery. A printer 11208 is an apparatus capable of printing various kinds of information relating to surgery in various forms such as a text, an image or a graph.

It is to be noted that the light source apparatus 11203 which supplies irradiation light when a surgical region is to be imaged to the endoscope 11100 may include a white light source which includes, for example, an LED, a laser light source or a combination of them. Where a white light source includes a combination of red, green, and blue (RGB) laser light sources, since the output intensity and the output timing can be controlled with a high degree of accuracy for each color (each wavelength), adjustment of the white balance of a picked up image can be performed by the light source apparatus 11203. Further, in this case, if laser beams from the respective RGB laser light sources are irradiated time-divisionally on an observation target and driving of the image pickup elements of the camera head 11102 are controlled in synchronism with the irradiation timings. Then images individually corresponding to the R, G and B colors can be also picked up time-divisionally. According to this method, a color image can be obtained even if color filters are not provided for the image pickup element.

Further, the light source apparatus 11203 may be controlled such that the intensity of light to be outputted is changed for each predetermined time. By controlling driving of the image pickup element of the camera head 11102 in synchronism with the timing of the change of the intensity of light to acquire images time-divisionally and synthesizing the images, an image of a high dynamic range free from underexposed blocked up shadows and overexposed highlights can be created.

Further, the light source apparatus 11203 may be configured to supply light of a predetermined wavelength band ready for special light observation. In special light observation, for example, by utilizing the wavelength dependency of absorption of light in a body tissue to irradiate light of a narrow band in comparison with irradiation light upon ordinary observation (namely, white light), narrow band observation (narrow band imaging) of imaging a predetermined tissue such as a blood vessel of a superficial portion of the mucous membrane or the like in a high contrast is performed. Alternatively, in special light observation, fluorescent observation for obtaining an image from fluorescent light generated by irradiation of excitation light may be performed. In fluorescent observation, it is possible to perform observation of fluorescent light from a body tissue by irradiating excitation light on the body tissue (autofluorescence observation) or to obtain a fluorescent light image by locally injecting a reagent such as indocyanine green (ICG) into a body tissue and irradiating excitation light corresponding to a fluorescent light wavelength of the reagent upon the body tissue. The light source apparatus 11203 can be configured to supply such narrow-band light and/or excitation light suitable for special light observation as described above.

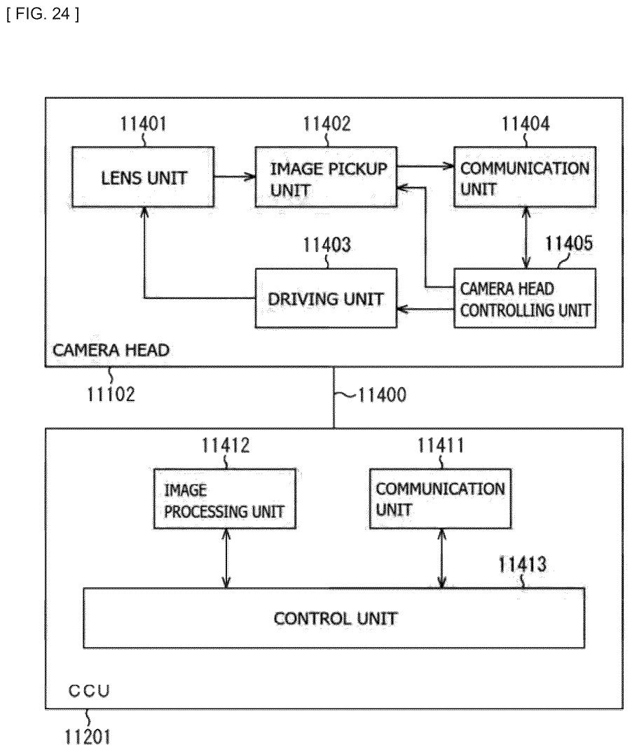

FIG. 24 is a block diagram depicting an example of a functional configuration of the camera head 11102 and the CCU 11201 depicted in FIG. 23.

The camera head 11102 includes a lens unit 11401, an image pickup unit 11402, a driving unit 11403, a communication unit 11404 and a camera head controlling unit 11405. The CCU 11201 includes a communication unit 11411, an image processing unit 11412 and a control unit 11413. The camera head 11102 and the CCU 11201 are connected for communication to each other by a transmission cable 11400.

The lens unit 11401 is an optical system, provided at a connecting location to the lens barrel 11101. Observation light taken in from a distal end of the lens barrel 11101 is guided to the camera head 11102 and introduced into the lens unit 11401. The lens unit 11401 includes a combination of a plurality of lenses including a zoom lens and a focusing lens.

The number of image pickup elements which is included by the image pickup unit 11402 may be one (single-plate type) or a plural number (multi-plate type). Where the image pickup unit 11402 is configured as that of the multi-plate type, for example, image signals corresponding to respective R, G and B are generated by the image pickup elements, and the image signals may be synthesized to obtain a color image. The image pickup unit 11402 may also be configured so as to have a pair of image pickup elements for acquiring respective image signals for the right eye and the left eye ready for three dimensional (3D) display. If 3D display is performed, then the depth of a living body tissue in a surgical region can be comprehended more accurately by the surgeon 11131. It is to be noted that, where the image pickup unit 11402 is configured as that of stereoscopic type, a plurality of systems of lens units 11401 are provided corresponding to the individual image pickup elements.

Further, the image pickup unit 11402 may not necessarily be provided on the camera head 11102. For example, the image pickup unit 11402 may be provided immediately behind the objective lens in the inside of the lens barrel 11101.

The driving unit 11403 includes an actuator and moves the zoom lens and the focusing lens of the lens unit 11401 by a predetermined distance along an optical axis under the control of the camera head controlling unit 11405. Consequently, the magnification and the focal point of a picked up image by the image pickup unit 11402 can be adjusted suitably.

The communication unit 11404 includes a communication apparatus for transmitting and receiving various kinds of information to and from the CCU 11201. The communication unit 11404 transmits an image signal acquired from the image pickup unit 11402 as RAW data to the CCU 11201 through the transmission cable 11400.

In addition, the communication unit 11404 receives a control signal for controlling driving of the camera head 11102 from the CCU 11201 and supplies the control signal to the camera head controlling unit 11405. The control signal includes information relating to image pickup conditions such as, for example, information that a frame rate of a picked up image is designated, information that an exposure value upon image picking up is designated and/or information that a magnification and a focal point of a picked up image are designated.

It is to be noted that the image pickup conditions such as the frame rate, exposure value, magnification or focal point may be designated by the user or may be set automatically by the control unit 11413 of the CCU 11201 on the basis of an acquired image signal. In the latter case, an auto exposure (AE) function, an auto focus (AF) function and an auto white balance (AWB) function are incorporated in the endoscope 11100.

The camera head controlling unit 11405 controls driving of the camera head 11102 on the basis of a control signal from the CCU 11201 received through the communication unit 11404.

The communication unit 11411 includes a communication apparatus for transmitting and receiving various kinds of information to and from the camera head 11102. The communication unit 11411 receives an image signal transmitted thereto from the camera head 11102 through the transmission cable 11400.

Further, the communication unit 11411 transmits a control signal for controlling driving of the camera head 11102 to the camera head 11102. The image signal and the control signal can be transmitted by electrical communication, optical communication or the like.

The image processing unit 11412 performs various image processes for an image signal in the form of RAW data transmitted thereto from the camera head 11102.

The control unit 11413 performs various kinds of control relating to image picking up of a surgical region or the like by the endoscope 11100 and display of a picked up image obtained by image picking up of the surgical region or the like. For example, the control unit 11413 creates a control signal for controlling driving of the camera head 11102.

Further, the control unit 11413 controls, on the basis of an image signal for which image processes have been performed by the image processing unit 11412, the display apparatus 11202 to display a picked up image in which the surgical region or the like is imaged. Thereupon, the control unit 11413 may recognize various objects in the picked up image using various image recognition technologies. For example, the control unit 11413 can recognize a surgical tool such as forceps, a particular living body region, bleeding, mist when the energy device 11112 is used and so forth by detecting the shape, color and so forth of edges of objects included in a picked up image. The control unit 11413 may cause, when it controls the display apparatus 11202 to display a picked up image, various kinds of surgery supporting information to be displayed in an overlapping manner with an image of the surgical region using a result of the recognition. Where surgery supporting information is displayed in an overlapping manner and presented to the surgeon 11131, the burden on the surgeon 11131 can be reduced and the surgeon 11131 can proceed with the surgery with certainty.

The transmission cable 11400 which connects the camera head 11102 and the CCU 11201 to each other is an electric signal cable ready for communication of an electric signal, an optical fiber ready for optical communication or a composite cable ready for both of electrical and optical communications.

Here, while, in the example depicted, communication is performed by wired communication using the transmission cable 11400, the communication between the camera head 11102 and the CCU 11201 may be performed by wireless communication.

Some examples of an endoscopic surgical system to which the technique of the present disclosure is applicable have been described above. Of the configuration described above, the technique of the present disclosure is applicable to the image pickup unit 11402. Application of the technique of the present disclosure improves an accuracy of the detection by the image pickup unit 11402.

It is to be noted that the endoscopic surgical system has been described herein as an example, but the technique of the present disclosure may be applied otherwise, for example, to a microsurgical system or the like.

### (Practical Application to Mobile Body)

The technique of the present disclosure is applicable to various products. For example, the technique of the present disclosure may be realized as a device mounted on any type of mobile body such as a car, an electric vehicle, a hybrid electric vehicle, a motorcycle, a bicycle, a personal mobility vehicle, an airplane, a drone, a ship, a robot, construction machinery, or agricultural machinery (tractor).

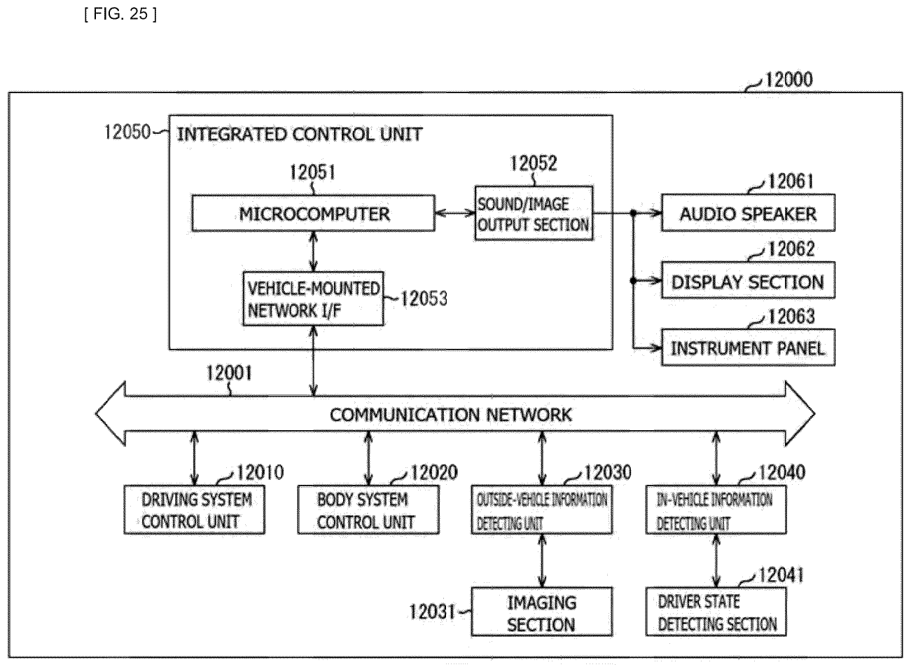

FIG. 25 is a block diagram depicting an example of schematic configuration of a vehicle control system as an example of a mobile body control system to which the technology according to an embodiment of the present disclosure can be applied.

The vehicle control system 12000 includes a plurality of electronic control units connected to each other via a communication network 12001. In the example depicted in FIG. 25, the vehicle control system 12000 includes a driving system control unit 12010, a body system control unit 12020, an outside-vehicle information detecting unit 12030, an in-vehicle information detecting unit 12040, and an integrated control unit 12050. In addition, a microcomputer 12051, a sound/image output section 12052, and a vehicle-mounted network interface (I/F) 12053 are illustrated as a functional configuration of the integrated control unit 12050.

The driving system control unit 12010 controls the operation of devices related to the driving system of the vehicle in accordance with various kinds of programs. For example, the driving system control unit 12010 functions as a control device for a driving force generating device for generating the driving force of the vehicle, such as an internal combustion engine, a driving motor, or the like, a driving force transmitting mechanism for transmitting the driving force to wheels, a steering mechanism for adjusting the steering angle of the vehicle, a braking device for generating the braking force of the vehicle, and the like.

The body system control unit 12020 controls the operation of various kinds of devices provided to a vehicle body in accordance with various kinds of programs. For example, the body system control unit 12020 functions as a control device for a keyless entry system, a smart key system, a power window device, or various kinds of lamps such as a headlamp, a backup lamp, a brake lamp, a turn signal, a fog lamp, or the like. In this case, radio waves transmitted from a mobile device as an alternative to a key or signals of various kinds of switches can be input to the body system control unit 12020. The body system control unit 12020 receives these input radio waves or signals, and controls a door lock device, the power window device, the lamps, or the like of the vehicle.

The outside-vehicle information detecting unit 12030 detects information about the outside of the vehicle including the vehicle control system 12000. For example, the outside-vehicle information detecting unit 12030 is connected with an imaging section 12031. The outside-vehicle information detecting unit 12030 makes the imaging section 12031 image an image of the outside of the vehicle, and receives the imaged image. On the basis of the received image, the outside-vehicle information detecting unit 12030 may perform processing of detecting an object such as a human, a vehicle, an obstacle, a sign, a character on a road surface, or the like, or processing of detecting a distance thereto.

The imaging section 12031 is an optical sensor that receives light, and which outputs an electric signal corresponding to a received light amount of the light. The imaging section 12031 can output the electric signal as an image, or can output the electric signal as information about a measured distance. In addition, the light received by the imaging section 12031 may be visible light, or may be invisible light such as infrared rays or the like.

The in-vehicle information detecting unit 12040 detects information about the inside of the vehicle. The in-vehicle information detecting unit 12040 is, for example, connected with a driver state detecting section 12041 that detects the state of a driver. The driver state detecting section 12041, for example, includes a camera that images the driver. On the basis of detection information input from the driver state detecting section 12041, the in-vehicle information detecting unit 12040 may calculate a degree of fatigue of the driver or a degree of concentration of the driver, or may determine whether the driver is dozing.

The microcomputer 12051 can calculate a control target value for the driving force generating device, the steering mechanism, or the braking device on the basis of the information about the inside or outside of the vehicle which information is obtained by the outside-vehicle information detecting unit 12030 or the in-vehicle information detecting unit 12040, and output a control command to the driving system control unit 12010. For example, the microcomputer 12051 can perform cooperative control intended to implement functions of an advanced driver assistance system (ADAS) which functions include collision avoidance or shock mitigation for the vehicle, following driving based on a following distance, vehicle speed maintaining driving, a warning of collision of the vehicle, a warning of deviation of the vehicle from a lane, or the like.

In addition, the microcomputer 12051 can perform cooperative control intended for automated driving, which makes the vehicle to travel automatedly without depending on the operation of the driver, or the like, by controlling the driving force generating device, the steering mechanism, the braking device, or the like on the basis of the information about the outside or inside of the vehicle which information is obtained by the outside-vehicle information detecting unit 12030 or the in-vehicle information detecting unit 12040.

In addition, the microcomputer 12051 can output a control command to the body system control unit 12020 on the basis of the information about the outside of the vehicle which information is obtained by the outside-vehicle information detecting unit 12030. For example, the microcomputer 12051 can perform cooperative control intended to prevent a glare by controlling the headlamp so as to change from a high beam to a low beam, for example, in accordance with the position of a preceding vehicle or an oncoming vehicle detected by the outside-vehicle information detecting unit 12030.

The sound/image output section 12052 transmits an output signal of at least one of a sound and an image to an output device capable of visually or auditorily notifying information to an occupant of the vehicle or the outside of the vehicle. In the example of FIG. 25, an audio speaker 12061, a display section 12062, and an instrument panel 12063 are illustrated as the output device. The display section 12062 may, for example, include at least one of an on-board display and a head-up display.

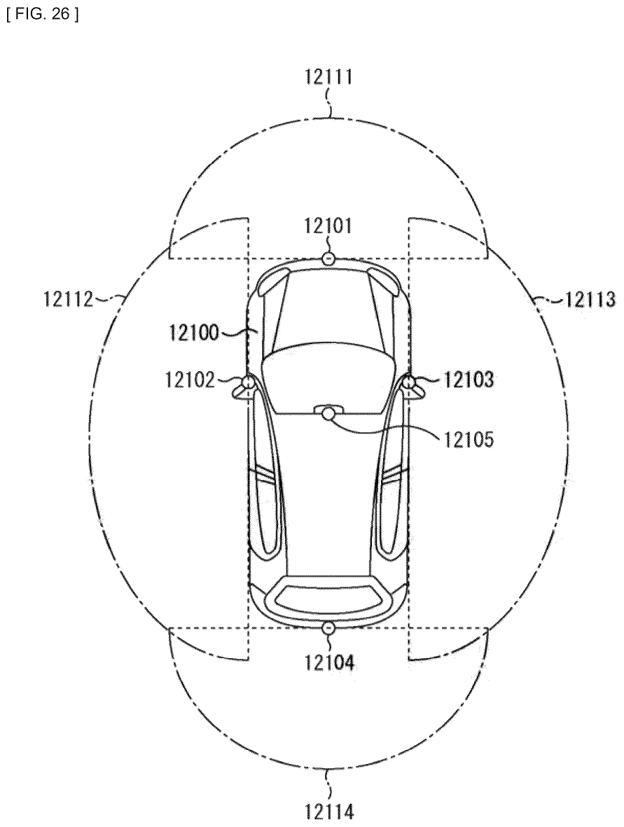

FIG. 26 is a diagram depicting an example of the installation position of the imaging section 12031.

In FIG. 26, the imaging section 12031 includes imaging sections 12101, 12102, 12103, 12104, and 12105.

The imaging sections 12101, 12102, 12103, 12104, and 12105 are, for example, disposed at positions on a front nose, sideview mirrors, a rear bumper, and a back door of the vehicle 12100 as well as a position on an upper portion of a windshield within the interior of the vehicle. The imaging section 12101 provided to the front nose and the imaging section 12105 provided to the upper portion of the windshield within the interior of the vehicle obtain mainly an image of the front of the vehicle 12100. The imaging sections 12102 and 12103 provided to the sideview mirrors obtain mainly an image of the sides of the vehicle 12100. The imaging section 12104 provided to the rear bumper or the back door obtains mainly an image of the rear of the vehicle 12100. The imaging section 12105 provided to the upper portion of the windshield within the interior of the vehicle is used mainly to detect a preceding vehicle, a pedestrian, an obstacle, a signal, a traffic sign, a lane, or the like.

Incidentally, FIG. 26 depicts an example of photographing ranges of the imaging sections 12101 to 12104. An imaging range 12111 represents the imaging range of the imaging section 12101 provided to the front nose. Imaging ranges 12112 and 12113 respectively represent the imaging ranges of the imaging sections 12102 and 12103 provided to the sideview mirrors. An imaging range 12114 represents the imaging range of the imaging section 12104 provided to the rear bumper or the back door. A bird's-eye image of the vehicle 12100 as viewed from above is obtained by superimposing image data imaged by the imaging sections 12101 to 12104, for example.

At least one of the imaging sections 12101 to 12104 may have a function of obtaining distance information. For example, at least one of the imaging sections 12101 to 12104 may be a stereo camera constituted of a plurality of imaging elements, or may be an imaging element having pixels for phase difference detection.

For example, the microcomputer 12051 can determine a distance to each three-dimensional object within the imaging ranges 12111 to 12114 and a temporal change in the distance (relative speed with respect to the vehicle 12100) on the basis of the distance information obtained from the imaging sections 12101 to 12104, and thereby extract, as a preceding vehicle, a nearest three-dimensional object in particular that is present on a traveling path of the vehicle 12100 and which travels in substantially the same direction as the vehicle 12100 at a predetermined speed (for example, equal to or more than 0 km/hour). Further, the microcomputer 12051 can set a following distance to be maintained in front of a preceding vehicle in advance, and perform automatic brake control (including following stop control), automatic acceleration control (including following start control), or the like. It is thus possible to perform cooperative control intended for automated driving that makes the vehicle travel automatedly without depending on the operation of the driver or the like.

For example, the microcomputer 12051 can classify three-dimensional object data on three-dimensional objects into three-dimensional object data of a two-wheeled vehicle, a standard-sized vehicle, a large-sized vehicle, a pedestrian, a utility pole, and other three-dimensional objects on the basis of the distance information obtained from the imaging sections 12101 to 12104, extract the classified three-dimensional object data, and use the extracted three-dimensional object data for automatic avoidance of an obstacle. For example, the microcomputer 12051 identifies obstacles around the vehicle 12100 as obstacles that the driver of the vehicle 12100 can recognize visually and obstacles that are difficult for the driver of the vehicle 12100 to recognize visually. Then, the microcomputer 12051 determines a collision risk indicating a risk of collision with each obstacle. In a situation in which the collision risk is equal to or higher than a set value and there is thus a possibility of collision, the microcomputer 12051 outputs a warning to the driver via the audio speaker 12061 or the display section 12062, and performs forced deceleration or avoidance steering via the driving system control unit 12010. The microcomputer 12051 can thereby assist in driving to avoid collision.

At least one of the imaging sections 12101 to 12104 may be an infrared camera that detects infrared rays. The microcomputer 12051 can, for example, recognize a pedestrian by determining whether or not there is a pedestrian in imaged images of the imaging sections 12101 to 12104. Such recognition of a pedestrian is, for example, performed by a procedure of extracting characteristic points in the imaged images of the imaging sections 12101 to 12104 as infrared cameras and a procedure of determining whether or not it is the pedestrian by performing pattern matching processing on a series of characteristic points representing the contour of the object. When the microcomputer 12051 determines that there is a pedestrian in the imaged images of the imaging sections 12101 to 12104, and thus recognizes the pedestrian, the sound/image output section 12052 controls the display section 12062 so that a square contour line for emphasis is displayed so as to be superimposed on the recognized pedestrian. The sound/image output section 12052 may also control the display section 12062 so that an icon or the like representing the pedestrian is displayed at a desired position.

Although some embodiments and Modification examples 1-6, application examples, and practical applications have been described above, the content of the present disclosure is not limited to the above embodiment, etc., but various modifications are possible. For example, in the above embodiment, the photoelectric conversion section 20 that detects green light, and the photoelectric conversion regions 32B and 32R that detect blue light and red light, respectively, are configured as an imaging element by stacking, but the content of the present disclosure is not limited to such a configuration. For example, the red or blue light may be detected in the photoelectric conversion section, or the green light may be detected in the photoelectric conversion region.

In addition, the number of these photoelectric conversion sections and photoelectric conversion regions and ratios thereof are not limited, and two or more photoelectric conversion sections may be provided, or a plurality of color signals may be acquired only by the photoelectric conversion section.

Furthermore, in the above embodiment, etc., as a plurality of electrodes included in the lower electrode 21, an example of a configuration in which two electrodes including a readout electrode 21A and a storage electrode 21B are provided or in which three electrodes including the readout electrode 21A, the storage electrode 21B, and the transfer electrode 21C or including the readout electrode 21A, the storage electrode 21B, and the shield electrode 21D are provided has been described. Other than this, four or more electrodes such as a discharge electrode or the like may be provided.

It is to be noted that the effects described herein are merely examples and are not limitative, and there may be other effects.

It is to be noted that it is possible for the present technique to have a configuration as follows. According to the present technique in the following configuration, a semiconductor layer including a first layer and a second layer is provided in which between an insulating layer covering, of a first electrode and a second electrode, the first electrode and having an opening above the second electrode and a photoelectric conversion layer, the first layer is formed at least above the first electrode, and the second layer is formed at least above the second electrode while being electrically coupled to the second electrode via the opening and having a difference from the first layer in at least one of material composition, crystallinity, impurity concentration contained, or constituent element. This improves a carrier transport characteristic to the second electrode, thereby making it possible to improve a captured image quality.

(1)

An imaging element including:
a first electrode and a second electrode;
a third electrode disposed opposed to the first electrode and the second electrode;
a photoelectric conversion layer provided between the first electrode and the third electrode and between the second electrode and the third electrode;
an insulating layer provided between the first electrode and the photoelectric conversion layer and between the second electrode and the photoelectric conversion layer, the insulating layer having an opening above the second electrode; and
a semiconductor layer including a first layer and a second layer, the first layer being provided between the photoelectric conversion layer and the insulating layer and formed at least above the first electrode, and the second layer being formed at least above the second electrode while being electrically coupled to the second electrode via the opening and having a difference from the first layer in at least one of material composition, crystallinity, impurity concentration contained, or constituent element.
   (2)

The imaging element according to (1), in which
an energy level at a lowest end of a conduction band of the second layer is equal to an energy level at a lowest end of a conduction band of the first layer, or is deeper than the energy level at the lowest end of the conduction band of the first layer.
   (3)

The imaging element according to (1) or (2), further including a first protective layer, the first protective layer including an inorganic material between the photoelectric conversion layer and the semiconductor layer.

(4)

The imaging element according to (3), in which
an energy level at a lowest end of a conduction band of the first layer is equal to an energy level at a lowest end of a conduction band of the first protective layer, or is deeper than the energy level at the lowest end of the conduction band of the first protective layer, and
an energy level at the lowest end of the conduction band of the first protective layer is equal to a LUMO of the photoelectric conversion layer, or is deeper than an energy level at a lowest end of a conduction band of the photoelectric conversion layer.
   (5)

The imaging element according to (3) or (4), in which
the second layer further includes a second protective layer, the second protective layer being provided along the opening and having an insulation property between the second layer and the first protective layer; and
an energy level at a lowest end of a conduction band of the second layer is shallower than an energy level at a lowest end of a conduction band of the second protective layer, the energy level at the lowest end of the conduction band of the second protective layer is shallower than a LUMO of the photoelectric conversion layer, and the LUMO of the photoelectric conversion layer is shallower than an energy level at a lowest end of a conduction band of the first protective layer.
   (6)

The imaging element according to any one of (1) to (5), in which
the first layer is a crystalline layer and the second layer is an amorphous layer.
   (7)

The imaging element according to any one of (1) to (6), in which
the first layer has the impurity concentration lower than the impurity concentration of the second layer.
   (8)

The imaging element according to any one of (1) to (7), in which
the first layer has the impurity concentration of one tenth or less of the impurity concentration of the second layer.
   (9)

The imaging element according to any one of (1) to (8), in which
an impurity contained in the first layer and the second layer is carbon.
   (10)

The imaging element according to any one of (1) to (9), in which
the semiconductor layer contains at least one type of element selected from indium, gallium, silicon, zinc, aluminum, and tin.
   (11)

The imaging element according to any one of (1) to (10), in which
the semiconductor layer includes IGZO, Ga₂O₃, GZO, IZO, ITO, InGaAlO, or InGaSiO.
   (12)

The imaging element according to any one of (3) to (11), in which
the first protective layer includes at least one type of element selected from tantalum, titanium, vanadium, niobium, tungsten, zirconium, hafnium, scandium, yttrium, lanthanum, gallium, and magnesium.
   (13)

The imaging element according to any one of (1) to (12), in which
the first electrode and the second electrode are disposed on an opposite side of a light incoming surface with respect to the photoelectric conversion layer.
   (14)

The imaging element according to any one of (1) to (13), in which
a voltage is applied separately to each of the first electrode and the second electrode.
   (15)

The imaging element according to any one of (1) to (14), further including a fourth electrode between the first electrode and the second electrode.

(16)

The imaging element according to (14) or (15), in which
one or a plurality of photoelectric conversion sections and one or a plurality of photoelectric conversion regions are stacked, the one or the plurality of photoelectric conversion sections including the first electrode, the second electrode, the third electrode, the photoelectric conversion layer, and the semiconductor layer, and the one or the plurality of photoelectric conversion regions performing photoelectric conversion on light having a different wavelength range from the photoelectric conversion section.
   (17)

The imaging element according to (16), in which
the one or the plurality of photoelectric conversion regions is formed embedded in a semiconductor substrate, and
the one or the plurality of photoelectric conversion sections is formed on a first surface side of the semiconductor substrate.
   (18)

A method of manufacturing an imaging element, the imaging element including
a first electrode and a second electrode,
a third electrode disposed opposed to the first electrode and the second electrode,
a photoelectric conversion layer provided between the first electrode and the third electrode and between the second electrode and the third electrode,
an insulating layer provided between the first electrode and the photoelectric conversion layer and between the second electrode and the photoelectric conversion layer, the insulating layer having an opening above the second electrode, and
a semiconductor layer including a first layer and a second layer, the first layer being provided between the photoelectric conversion layer and the insulating layer and formed at least above the first electrode, and the second layer being formed at least above the second electrode while being electrically coupled to the second electrode via the opening and having a difference from the first layer in at least one of material composition, crystallinity, impurity concentration contained, or constituent element,
the method including:
forming the first layer using a physical vapor deposition method; and
forming the second layer using an atomic layer deposition method.
   (19)

A light detection device that includes a plurality of pixels each including one or a plurality of imaging elements, the one or the plurality of imaging elements including:
a first electrode and a second electrode;
a third electrode disposed opposed to the first electrode and the second electrode;
a photoelectric conversion layer provided between the first electrode and the third electrode and between the second electrode and the third electrode;
an insulating layer provided between the first electrode and the photoelectric conversion layer and between the second electrode and the photoelectric conversion layer, the insulating layer having an opening above the second electrode; and
a semiconductor layer including a first layer and a second layer, the first layer being provided between the photoelectric conversion layer and the insulating layer and formed at least above the first electrode, and the second layer being formed at least above the second electrode while being electrically coupled to the second electrode via the opening and having a difference from the first layer in at least one of material composition, crystallinity, impurity concentration contained, or constituent element.

The present application claims the benefit of Japanese Priority Patent Application JP2022-050525 filed with the Japan Patent Office on Mar. 25, 2022, the entire contents of which are incorporated herein by reference.

It should be understood by those skilled in the art that various modifications, combinations, sub-combinations and alterations may occur depending on design requirements and other factors insofar as they are within the scope of the appended claims or the equivalents thereof.

1. An imaging element comprising:
a first electrode and a second electrode;
a third electrode disposed opposed to the first electrode and the second electrode;
a photoelectric conversion layer provided between the first electrode and the third electrode and between the second electrode and the third electrode;
an insulating layer provided between the first electrode and the photoelectric conversion layer and between the second electrode and the photoelectric conversion layer, the insulating layer having an opening above the second electrode; and
a semiconductor layer including a first layer and a second layer, the first layer being provided between the photoelectric conversion layer and the insulating layer and formed at least above the first electrode, and the second layer being formed at least above the second electrode while being electrically coupled to the second electrode via the opening and having a difference from the first layer in at least one of material composition, crystallinity, impurity concentration contained, or constituent element.

2. The imaging element according to claim 1, wherein
an energy level at a lowest end of a conduction band of the second layer is equal to an energy level at a lowest end of a conduction band of the first layer, or is deeper than the energy level at the lowest end of the conduction band of the first layer.

3. The imaging element according to claim 1, further comprising a first protective layer, the first protective layer including an inorganic material between the photoelectric conversion layer and the semiconductor layer.

4. The imaging element according to claim 3, wherein
an energy level at a lowest end of a conduction band of the first layer is equal to an energy level at a lowest end of a conduction band of the first protective layer, or is deeper than the energy level at the lowest end of the conduction band of the first protective layer, and
an energy level at the lowest end of the conduction band of the first protective layer is equal to a LUMO of the photoelectric conversion layer, or is deeper than an energy level at a lowest end of a conduction band of the photoelectric conversion layer.

5. The imaging element according to claim 3, wherein
the second layer further includes a second protective layer, the second protective layer being provided along the opening and having an insulation property between the second layer and the first protective layer; and
an energy level at a lowest end of a conduction band of the second layer is shallower than an energy level at a lowest end of a conduction band of the second protective layer, the energy level at the lowest end of the conduction band of the second protective layer is shallower than a LUMO of the photoelectric conversion layer, and the LUMO of the photoelectric conversion layer is shallower than an energy level at a lowest end of a conduction band of the first protective layer.

6. The imaging element according to claim 1, wherein
the first layer comprises a crystalline layer and the second layer comprises an amorphous layer.

7. The imaging element according to claim 1, wherein
the first layer has the impurity concentration lower than the impurity concentration of the second layer.

8. The imaging element according to claim 1, wherein
the first layer has the impurity concentration of one tenth or less of the impurity concentration of the second layer.

9. The imaging element according to claim 1, wherein
an impurity contained in the first layer and the second layer comprises carbon.

10. The imaging element according to claim 1, wherein
the semiconductor layer contains at least one type of element selected from indium, gallium, silicon, zinc, aluminum, and tin.

11. The imaging element according to claim 1, wherein
the semiconductor layer includes IGZO, Ga₂O₃, GZO, IZO, ITO, InGaAlO, or InGaSiO.

12. The imaging element according to claim 3, wherein
the first protective layer includes at least one type of element selected from tantalum, titanium, vanadium, niobium, tungsten, zirconium, hafnium, scandium, yttrium, lanthanum, gallium, and magnesium.

13. The imaging element according to claim 1, wherein
the first electrode and the second electrode are disposed on an opposite side of a light incoming surface with respect to the photoelectric conversion layer.

14. The imaging element according to claim 1, wherein
a voltage is applied separately to each of the first electrode and the second electrode.

15. The imaging element according to claim 1, further comprising a fourth electrode between the first electrode and the second electrode.

16. The imaging element according to claim 14, wherein
one or a plurality of photoelectric conversion sections and one or a plurality of photoelectric conversion regions are stacked, the one or the plurality of photoelectric conversion sections including the first electrode, the second electrode, the third electrode, the photoelectric conversion layer, and the semiconductor layer, and the one or the plurality of photoelectric conversion regions performing photoelectric conversion on light having a different wavelength range from the photoelectric conversion section.

17. The imaging element according to claim 16, wherein
the one or the plurality of photoelectric conversion regions is formed embedded in a semiconductor substrate, and
the one or the plurality of photoelectric conversion sections is formed on a first surface side of the semiconductor substrate.

18. A method of manufacturing an imaging element, the imaging element comprising
a first electrode and a second electrode,
a third electrode disposed opposed to the first electrode and the second electrode,
a photoelectric conversion layer provided between the first electrode and the third electrode and between the second electrode and the third electrode,
an insulating layer provided between the first electrode and the photoelectric conversion layer and between the second electrode and the photoelectric conversion layer, the insulating layer having an opening above the second electrode, and
a semiconductor layer including a first layer and a second layer, the first layer being provided between the photoelectric conversion layer and the insulating layer and formed at least above the first electrode, and the second layer being formed at least above the second electrode while being electrically coupled to the second electrode via the opening and having a difference from the first layer in at least one of material composition, crystallinity, impurity concentration contained, or constituent element,
the method comprising:
forming the first layer using a physical vapor deposition method; and
forming the second layer using an atomic layer deposition method.

19. A light detection device that includes a plurality of pixels each including one or a plurality of imaging elements, the one or the plurality of imaging elements comprising:
a first electrode and a second electrode;
a third electrode disposed opposed to the first electrode and the second electrode;
a photoelectric conversion layer provided between the first electrode and the third electrode and between the second electrode and the third electrode;
an insulating layer provided between the first electrode and the photoelectric conversion layer and between the second electrode and the photoelectric conversion layer, the insulating layer having an opening above the second electrode; and
a semiconductor layer including a first layer and a second layer, the first layer being provided between the photoelectric conversion layer and the insulating layer and formed at least above the first electrode, and the second layer being formed at least above the second electrode while being electrically coupled to the second electrode via the opening and having a difference from the first layer in at least one of material composition, crystallinity, impurity concentration contained, or constituent element.
